(19) United States
(12) Patent Application Publication
(10) Pub. No.: US 2025/0352050 A1
(43) Pub. Date: Nov. 20, 2025

(54) SYSTEM FOR STABILIZED NONINVASIVE IMAGING OF MICROVASCULATURE IN THE ORAL MUCOSA

(71) Applicant: The General Hospital Corporation, Boston, MA (US)

(72) Inventors: Arutyun Bagramyan, Boston, MA (US); Charles Lin, Boston, MA (US); Juwell W. Wu, Boston, MA (US)

(21) Appl. No.: 18/867,629

(22) PCT Filed: May 23, 2023
(86) PCT No.: PCT/US2023/067342

§ 371 Date: Nov. 20, 2024

Related U.S. Application Data
(60) Provisional application No. 63/344,975, filed on May 23, 2022.

(51) Int. Cl. A61B 1/24; A61B 1/00; A61B 1/05; A61B 1/06

(52) U.S. Cl. CPC A61B 1/24; A61B 1/00124; A61B 1/05; A61B 1/0627; A61B 2560/04; A61B 2576/02

(57) ABSTRACT

Disclosed is a system for imaging of microvasculature of tissue of a subject. The system can comprise: (a) a tissue stabilizer structured to contact the tissue of the subject to maintain a position of the region of microvasculature being imaged, and (b) an imaging instrument including: (i) a housing having an imaging section, (ii) an illumination device having a light-outputting end positioned in the imaging section for illuminating a region of the microvasculature with light, wherein the light-outputting end is offset relative to an optical axis of the imaging section; (iii) an objective lens positioned in the imaging section such that the objective lens receives at least a portion of light scattered by the region of the microvasculature, and (iv) an image detector positioned in the imaging section such that the image detector receives light redirected by the objective lens and detects microscopic images of the region of microvasculature.

### CROSS-REFERENCE TO RELATED APPLICATIONS

This application is based on, claims benefit of, and claims priority to U.S. Application No. 63/344,975, filed on May 23, 2022, which is hereby incorporated by reference herein in its entirety for all purposes.

### STATEMENT REGARDING FEDERALLY SPONSORED RESEARCH

This invention was made with government support under FA9550-20-1-0063 awarded by the Air Force Office of Scientific Research. The government has certain rights in the invention.

### BACKGROUND OF THE INVENTION

### 1. Field of the Invention

The invention relates to a system and methods for imaging of microvasculature of tissue of a subject, and more particularly to a system and methods for stabilized noninvasive imaging of microvasculature in the oral mucosa.

### 2. Background and Description of the Related Art

Sepsis is a life-threatening medical emergency that affects more than 30 million people worldwide and takes 8 million lives, including more than 3 million children. It is the number one cause of mortality for hospitalized patients in the US, accounting for ˜50% of deaths in the intensive care unit. Early diagnosis of sepsis is critical, as every hour of delay is estimated to increase the mortality rate by 7-10% (see Farkas, J Thorac Dis 2020; 12(Suppl 1): S16-S21). The white blood cell (leukocyte) count is among the most used diagnostic parameters for guiding interventions in patients affected by sepsis. However, the current standard of measuring blood cells is invasive and requires repeated blood draws from a vulnerable population of patients at high risk of medical complications (secondary infections, anemia, chronic pain, etc.). In addition, laboratory analysis takes time, and clinical hemocytometers are not always available in resource-poor settings.

Though well-characterized in animal models using intravital microscopy, the rolling and adhesion events of leukocytes (collectively known as leukocyte-endothelial interaction, or LEI) have rarely been observed in humans. Conceptually, imaging cell motion as a potential source of diagnostic information has yet to be explored in clinics, as traditional histopathology has relied on the static examination of biopsied samples. LEI is reported to be significantly increased in the sublingual microvasculature of patients with systemic inflammation such as sepsis and ischemia-reperfusion injury. However, assessing LEI in clinical settings has been challenging due to the lack of proper detection and analytical tools.

Therefore, there is a need for improved systems and methods for imaging of microvasculature of a subject such that observed leukocyte-endothelial interaction can provide a source of diagnostic information.

### SUMMARY OF THE INVENTION

To address these limitations, we have developed a system for imaging of microvasculature of tissue wherein the system includes a tissue stabilizer and an imaging instrument (e.g., miniaturized microscope) for real-time, non-invasive label-free detection and quantification of blood cells in vivo by means of phase-gradient microscopy with oblique back illumination. With this system, we can capture videos of fast-moving blood cells in the flowing stream as well as slow-moving leukocytes that are rolling and adhering to the vascular wall of the buccal microvasculature of healthy human volunteers. Notably, leukocyte rolling and adhesion are new diagnostic parameters based on cell dynamics (motion) rather than traditional static parameters such as cell morphology and are therefore only obtainable using in vivo microscopy. Disclosed for providing clinicians with reliable and actionable results is a custom algorithm for automated quantification, movement analysis and potential classification of different types of leukocytes.

In one aspect, the disclosure provides a system for imaging of microvasculature of tissue of a subject. The system comprises: (a) a tissue stabilizer structured to contact the tissue of the subject to maintain a position of the region of the microvasculature being imaged; and (b) an imaging instrument including: (i) a housing having an imaging section, (ii) an illumination device having a light-outputting end positioned in the imaging section of the housing for illuminating a region of the microvasculature with light, wherein the light-outputting end is offset relative to an optical axis of the imaging section, (iii) an objective lens positioned in the imaging section of the housing such that the objective lens receives at least a portion of light scattered by the region of the microvasculature, and (iv) an image detector positioned in the imaging section of the housing such that the image detector receives light redirected by the objective lens and detects microscopic images of the region of the microvasculature.

In one embodiment, the tissue stabilizer comprises a base, a sliding mechanism mounted on the base, an adapter for contacting the tissue, the adapter being mounted on the sliding mechanism, and the adapter is moveable toward and away from the base. The base can comprise a chin holder, and the tissue stabilizer further comprises a frame, the chin holder and a forehead holder being mounted on the frame. The adapter can include a patterned surface finish for contacting the tissue. The adapter can include opposed tabs for contacting the tissue. The adapter can apply mechanical pressure on edges of the oral mucosa tissue, at a distance of at least two millimeters (e.g., five millimeters) from imaging regions of interest to minimize impact of the mechanical pressure on an imaging area. In one embodiment, the adapter is bendable. In one embodiment, the adapter is rigid. In one embodiment, the adapter includes single or multiple light sources. In one embodiment, the adapter includes single or multiple optical elements. In one embodiment, the adapter includes a vacuum system. In one embodiment, the adapter further includes a transparent sheet mounted between the opposed tabs.

In one embodiment, the tissue stabilizer comprises a base, a sliding mechanism mounted on the base, an adapter for contacting the tissue, the adapter being mounted on the sliding mechanism, and the adapter is moveable laterally with respect to the base. In one embodiment, the adapter is dimensioned for contacting oral mucosa of the subject. In one embodiment, the adapter is dimensioned for contacting a lip of the subject. In one embodiment, the adapter comprises a rod mounted between opposed connectors, the rod being dimensioned for contacting the tissue. In one embodiment, the adapter comprises a flexible loop, the rod being dimensioned for contacting the tissue.

In one embodiment, the objective lens is a microlens. In one embodiment, the objective lens is a gradient index (GRIN) objective lens. In one embodiment, the objective lens is a gradient index (GRIN) objective lens, and the system further comprises a doublet achromat lens. In one embodiment, the image detector is a camera. In one embodiment, the image detector is a CMOS sensor. In one embodiment, the image detector is moveable with respect to the objective lens. In one embodiment, the image detector detects microscopic images using oblique back-illumination (OBM). In one embodiment, the image detector detects microscopic images using offset trans-illumination (OTM). In one embodiment, the imaging section further comprises a vacuum device for stabilizing the tissue being imaged. In one embodiment, the imaging section further comprises an irrigation channel for supplying a fluid to keep the tissue being imaged moist. In one embodiment, the illumination device comprises a light source and an optical fiber having the light-outputting end. In one embodiment, the imaging section further comprises an imaging tip that contains the objective lens, a vacuum device, an irrigation channel, and an illumination fiber of the illumination device, and the objective lens is a microlens. In one embodiment, the imaging section further comprises an imaging tip that contains the objective lens, a vacuum device, an irrigation channel, and an illumination fiber of the illumination device, and the objective lens is gradient index (GRIN) lens. The imaging tip can be disposable.

In one embodiment, the microscopic images include images of leukocyte-endothelial interaction in the microvasculature. In one embodiment, the imaging is label-free imaging. In one embodiment, the microscopic images are phase-gradient contrast images. In one embodiment, the illumination device comprises a light source and an optical fiber having the light-outputting end, and the light source comprises a light-emitting diode. In one embodiment, the imaging is at a frame rate of 1 Hz to 1000 Hz. In one embodiment, the imaging is at a frame rate of 1 Hz to 300 Hz.

In one embodiment of the system, injected light power is automatically adjusted by a controller to prevent pixel(s) saturation of a data acquisition element (e.g., CMOS). In one embodiment of the system, scattered light collection time (exposure time) of a data acquisition element is automatically adjusted by software to prevent pixel(s) saturation. In one embodiment of the system, the microscopic images include images of leukocytes in the microvasculature, and the system further comprises a controller in electrical communication with the illumination device and the image detector, the controller being configured to execute a program stored in the controller to: (i) receive the microscopic images from the image detector, and (ii) use automated frame-by-frame leukocyte tracking to calculate average rolling velocity of the leukocytes in the microvasculature. In one embodiment, the controller executes the program stored in the controller to: (iii) compare the average rolling velocity of the leukocytes in the microvasculature to an average rolling velocity of leukocytes in heathy tissue.

In yet another aspect, the disclosure provides a system for imaging of microvasculature of tissue of a subject. The system comprises: an imaging instrument including a housing having an imaging section; a tissue stabilizer structured to contact the tissue of the subject to maintain a position of the region of the microvasculature being imaged by the imaging instrument; an illumination device having a light-outputting end positioned in the tissue stabilizer for illuminating a region of the microvasculature with light; an objective lens positioned in the imaging section of the housing such that the objective lens receives at least a portion of light scattered by the region of the microvasculature; and an image detector positioned in the imaging section of the housing such that the image detector receives light redirected by the objective lens and detects microscopic images of the region of the microvasculature.

In one embodiment of the system, the tissue stabilizer comprises a base, a sliding mechanism mounted on the base, an adapter for contacting the tissue, the adapter being mounted on the sliding mechanism, and the adapter is moveable toward and away from the base. In one embodiment, the light-outputting end of the illumination device is positioned in the adapter. In one embodiment, the base comprises a chin holder, and the light-outputting end of the illumination device is positioned in the chin holder.

In still another aspect, the disclosure provides a system for imaging of microvasculature of tissue of a subject. The system comprises: a tissue stabilizer structured to contact the tissue of the subject to maintain a position of the region of the microvasculature being imaged; an illumination device having a light-outputting end positioned in the tissue stabilizer for illuminating a region of the microvasculature with light; an objective lens positioned in the tissue stabilizer such that the objective lens receives at least a portion of light scattered by the region of the microvasculature; and an image detector positioned in the tissue stabilizer such that the image detector receives light redirected by the objective lens and detects microscopic images of the region of the microvasculature. In one embodiment of the system, the tissue stabilizer comprises a first arm and an opposed second arm, the first arm and the second arm defining a space therebetween for receiving the tissue, and the illumination device, the objective lens, and the image detector are arranged on the first arm such that the image detector detects microscopic images using oblique back-illumination (OBM). In one embodiment of the system, the tissue stabilizer comprises a first arm and an opposed second arm, the first arm and the second arm defining a space therebetween for receiving the tissue, and the objective lens and the image detector are arranged on the first arm, and the illumination device is arranged on the second arm such that the image detector detects microscopic images using offset trans-illumination (OTM). In one embodiment of the system, the tissue stabilizer comprises a first arm, an opposed second arm, and a hinge connecting the first arm and the second arm such that a variable size space is created between the first arm and the second arm for receiving the tissue.

In yet another aspect, the disclosure provides a system for imaging of microvasculature of tissue of a subject. The system comprises: an imaging instrument operable to capture an image; an electronic processor in communication with the imaging instrument, the electronic processor being configured to execute a program stored in the electronic processor to: receive the image from the imaging instrument; and reduce a foreground of the image to a skeleton that captures one or more attributes of the foreground including at least one of curvature, connectivity, and extent, wherein the skeleton is used to define a transformed coordinate system for quantifying one or more perfusion parameters in the microvasculature. In one embodiment, the skeleton is a line that follows an axis of a vessel of the microvasculature and bends in accordance with local curvature of the vessel. In one embodiment of the system, the electronic processor executes the program stored in the electronic processor to: create a transformed coordinate system by generating multiple gridlines to cover a full width of a region of interest (ROI) of microvasculature. In one embodiment of the system, the electronic processor executes the program stored in the electronic processor to: create the transformed coordinate system such that two axes run parallel and normal to blood flow, respectively, wherein an axis parallel to the blood flow is defined by the skeleton, and an axis normal to the blood flow is defined by normal lines of the skeleton. In one embodiment of the system, the electronic processor executes the program stored in the electronic processor to: create a collection of skeleton and lines created in reference to the skeleton defining an x′-axis and y′-gridlines of the transformed coordinate system, the y′-gridlines running in the direction of blood flow of microvascular ROI. In one embodiment of the system, the y′-gridlines of the transformed coordinate system have a same pixel length, regardless of curvature of the ROI. In one embodiment of the system, the electronic processor executes the program stored in the electronic processor to: draw a space-time diagram for each y′-gridline at each time segment and vessel block, the vessel block being defined as a unit for length along an axis of microvascular ROI. In one embodiment of the system, the electronic processor executes the program stored in the electronic processor to: calculate a blood flow velocity by consolidating multiple space-time diagrams of individual y′-gridline, time segment and vessel block. In one embodiment of the system, the electronic processor executes the program stored in the electronic processor to: calculate blood flow volume rate by multiplying the blood flow velocity and a cross-section area of the ROI. In one embodiment of the system, the electronic processor executes the program stored in the electronic processor to: calculate a count of leukocytes by summing along slopes of the space-time diagrams to generate an intensity profile wherein the intensity profiles are further consolidated from the multiple y′-gridlines, time segments and vessel blocks such that a number of peaks in a consolidated intensity profile gives an estimate of the count of leukocytes. In one embodiment, the consolidation is performed by dynamic time warping to peak match an intensity profile of vessel blocks, while allowing variations in time delay among candidate leukocytes. In one embodiment of the system, the electronic processor executes the program stored in the electronic processor to: estimate a time and spatial location of appearance (“gate”) of candidate leukocytes by determining a peak position in an intensity profile. In one embodiment of the system, the electronic processor executes the program stored in the electronic processor to: gate an approximate space and time of appearance of candidate leukocytes in the video using a consolidated intensity profile.

In still another aspect, the disclosure provides a system for imaging of microvasculature of tissue of a subject. The system comprises: an imaging instrument operable to capture an image; an electronic processor in communication with the imaging instrument, the electronic processor being configured to execute a program stored in the electronic processor to: receive the image from the imaging instrument; access a deep learning model that has been trained on training data to detect perfusion and leukocyte feature data from the image input; and apply the image to the machine learning model to quantify one or more perfusion parameters in the microvasculature. In one embodiment of the system, the deep learning model is a neural network. In one embodiment of the system, the neural network is a convolutional neural network. In one embodiment of the system, the machine learning model is applied to restricted spatial regions and time (“gates”) that contain candidate leukocytes. In one embodiment of the system, the electronic processor executes the program stored in the electronic processor to: detect coordinates of the image at which a leukocyte is detected, and a probability score of the detection.

In yet another aspect, the disclosure provides a method for in vivo flow cytometry of a biological fluid in a subject. The method can comprise: (a) contacting tissue of the subject with a tissue stabilizer to maintain a position of a biological structure of the subject; (b) providing, using an illumination device, light to a portion of a region of the biological structure to continuously illuminate the region of the biological structure; (c) continuously detecting, using an image detector, microscopic images from the region of the biological structure based on light scattered by the biological structure of the subject, wherein the light-outputting end is offset relative to an optical axis of the imaging section; and (d) analyzing the microscopic images to identify characteristics of a biological fluid in the biological structure. In one embodiment of the method, step (c) comprises detecting the microscopic images comprises producing optical images through oblique back-illumination microscopy (OBM). In one embodiment of the method, step (c) comprises detecting the microscopic images comprises producing optical images through offset trans-illumination (OTM).

In one embodiment of the method, the biological structure is microvasculature of the subject; and step (d) comprises quantifying one or more perfusion parameters in the microvasculature.

In one embodiment of the method, the biological structure is microvasculature of the subject; and step (d) comprises quantifying a count of leukocytes in the microvasculature.

In one embodiment of the method, step (c) comprises detecting the microscopic images without a label. In one embodiment of the method, step (c) comprises detecting the microscopic images at a frame rate of 1 Hz to 1000 Hz.

In one embodiment of the method, the biological structure is microvasculature of the subject; and step (d) comprises using automated frame-by-frame leukocyte tracking to calculate average rolling velocity of leukocytes in the microvasculature.

In one embodiment of the method, the biological structure is microvasculature of the subject; and step (d) comprises reducing a foreground of each microscopic image to a skeleton that captures one or more attributes of the foreground including at least one of curvature, connectivity, and extent, the skeleton for use in quantifying one or more perfusion parameters in the microvasculature.

In one embodiment of the method, the biological structure is microvasculature of the subject; and step (d) further comprises creating a transformed coordinate system by generating multiple gridlines to cover a full width of a region of interest (ROI) of microvasculature.

In one embodiment of the method, the biological structure is microvasculature of the subject; and step (d) further comprises creating the transformed coordinate system in which two axes run parallel and normal to blood flow, respectively, wherein an axis parallel to the blood flow is defined by the skeleton, and an axis normal to the blood flow is defined by normal lines of the skeleton.

In one embodiment of the method, the biological structure is microvasculature of the subject; and step (d) comprises creating a transformed coordinate system wherein two axes run parallel and normal to the blood flow, respectively.

In one embodiment of the method, step (d) comprises creating the transformed coordinate system wherein an axis parallel to the blood flow is defined by the skeleton, and an axis normal to the blood flow is defined by normal lines of the skeleton.

In one embodiment of the method, the biological structure is microvasculature of the subject; and step (d) comprises drawing a space-time diagram for each y′-gridline at each time segment and vessel block, the vessel block being defined as a unit for length along an axis of microvascular of the ROI.

In one embodiment of the method, step (d) further comprises calculating a blood flow velocity by consolidating multiple space-time diagrams of individual y′-gridline, time segment and vessel block.

In one embodiment of the method, the biological structure is microvasculature of the subject; and step (d) comprises accessing a deep learning model that has been trained on training data to detect perfusion and leukocyte feature data from the image input; and applying the image to the machine learning model to quantify one or more perfusion parameters in the microvasculature.

These and other features, aspects and advantages of various embodiments of the present disclosure will become better understood with regard to the following description, appended claims, and accompanying Figures.

### BRIEF DESCRIPTION OF THE DRAWINGS

FIG. 1A is a side view of one example embodiment of a bench-top tissue stabilizer according to the invention positioned on a human subject.

FIG. 1B is a side view of one example embodiment of a portable tissue stabilizer according to the invention positioned on a human subject.

FIG. 2 is another side view of the bench-top tissue stabilizer of FIG. 1A.

FIG. 3A is a rear view of one example embodiment of a multicomponent tissue stabilizer according to the invention with the adapter noted by a dashed rectangle.

FIG. 3B is a rear view of the multicomponent tissue stabilizer of FIG. 3A with the adapter holders noted by dashed rectangles.

FIG. 3C is a rear view of the multicomponent tissue stabilizer of FIG. 3A with components of the sliding mechanism noted by dashed rectangles.

FIG. 3D is a rear view of the multicomponent tissue stabilizer of FIG. 3A with the chin holder and forehead holder noted by a dashed rectangle.

FIG. 4A is a side view of one example embodiment of a chin holder of a tissue stabilizer according to the invention positioned on a human subject.

FIG. 4B is a side view of one example embodiment of a chin holder of a tissue stabilizer according to the invention positioned on a human subject.

FIG. 5 is a rear view of one example embodiment of a multicomponent tissue stabilizer according to the invention.

FIG. 6A is a rear view of the multicomponent tissue stabilizer of FIG. 5 with components of the sliding mechanism depicting vertical sliding movement.

FIG. 6B is a rear view of the multicomponent tissue stabilizer of FIG. 5 with components of the sliding mechanism depicting horizontal sliding movement.

FIG. 6C is a rear view of the multicomponent tissue stabilizer of FIG. 5 with components of the sliding mechanism depicting fixation screws.

FIG. 7A is a perspective view of one example embodiment of an adapter of a tissue stabilizer according to the invention.

FIG. 7B is a side view of the adapter of FIG. 7A.

FIG. 7C is another perspective view of the adapter of FIG. 7A.

FIG. 8A is a front view of the multicomponent tissue stabilizer of FIG. 5 during use with a human subject.

FIG. 8B is a detailed view of FIG. 8A of the oral mucosa tissue area, in which the microvasculature is exposed for investigation and imaging.

FIG. 9A is a perspective view of another example embodiment of an adapter of a tissue stabilizer according to the invention.

FIG. 9B is a front view of the adapter of FIG. 9A during use with a human subject in which the microvasculature is exposed for future investigation and imaging.

FIG. 10A is a perspective view of another example embodiment of an adapter of a tissue stabilizer according to the invention.

FIG. 10B is a front view of the adapter of FIG. 10A during use with a human subject, in which the microvasculature is exposed for future investigation and imaging.

FIG. 11A is a perspective view of another example embodiment of an adapter of a tissue stabilizer according to the invention.

FIG. 11B is a front view of the adapter of FIG. 10A during use with a human subject, in which the microvasculature is exposed for future investigation and imaging.

FIG. 12A are side views of a method of using of the bench-top tissue stabilizer of FIG. 1A.

FIG. 12B are side views of a method of using of the portable tissue stabilizer of FIG. 1B.

FIG. 13A is a side view of a method of using of the bench-top tissue stabilizer of FIG. 1A in a system for imaging of microvasculature of tissue of a subject according to the invention.

FIG. 13B is a side view of a method of using of the portable tissue stabilizer of FIG. 1B on a human subject.

FIG. 14A is a top view of one example embodiment a tissue stabilizer with light sources integrated into the chin holder according to the invention.

FIG. 14B is a perspective view of one example embodiment of a tissue stabilizer with light sources integrated into the adapter according to the invention.

FIG. 15A is a side view of components of an example embodiment of a bench-top tissue stabilizer according to the invention positioned on a human subject and an imaging instrument.

FIG. 15B is a perspective view of imaging instrument of the system for imaging of microvasculature of tissue of a subject of FIG. 15A.

FIG. 16 is a perspective view of the imaging instrument of FIG. 15B.

FIG. 17 is a side view of the imaging instrument of FIG. 15B.

FIG. 18A is an optical design of a light collection pathway of an example embodiment of an imaging instrument of a system according to the invention.

FIG. 18B is another optical design of a light collection pathway of another example embodiment of an imaging instrument of a system according to the invention.

FIG. 19A shows an example light injection configuration and imaging modality of an imaging instrument which includes a GRIN lens objective (1) and light source/optical fiber (2) and uses Oblique Back-Illumination (OBM).

FIG. 19B shows another example light injection configuration and imaging modality of an imaging instrument which includes a GRIN lens objective (1) and light source/optical fiber (2) and uses Offset Trans-Illumination (OTM).

FIG. 20A is a perspective view of another example embodiment of an imaging instrument of a system according to the invention.

FIG. 20B is a front view of the tip of the imaging instrument of FIG. 20A without components.

FIG. 20C is a front view of the tip of the imaging instrument of FIG. 20A with components.

FIG. 21A is an exploded perspective view of another example embodiment of an imaging instrument according to the invention with a disposable tip.

FIG. 21B is a perspective view of the imaging instrument of FIG. 21A with the disposable tip clipped.

FIG. 22 is a detailed perspective view of the imaging instrument of FIG. 21A with the disposable tip clipped.

FIG. 23A is a side view of another example embodiment of a system for imaging of microvasculature of tissue according to the invention positioned on a human subject.

FIG. 23B is a perspective view of the portable system of FIG. 23A.

FIG. 23C is a side view of one embodiment of the portable system of FIGS. 23A and 23B in an oblique back-illumination configuration.

FIG. 23D is a side view of another embodiment of the portable system of FIGS. 23A and 23B in an offset trans-illumination configuration.

FIG. 24A is a side view of another example embodiment of a portable system for imaging of microvasculature of tissue according to the invention positioned on a human subject.

FIG. 24B is a perspective view of the portable system of FIG. 24A.

FIG. 25A is a side view of the tissue stabilizer of the system of FIG. 24A.

FIG. 25B is a side view of the imaging instrument of the system of FIG. 24A.

FIG. 25C is a front view of the tip of the tissue stabilizer of the system of FIG. 24A.

FIG. 26 is a side view of components of another example embodiment of a system for imaging of microvasculature of tissue according to the invention that uses a microscope objective positioned on a human subject.

FIG. 27 shows leukocyte-endothelial interaction (LEI) in human microvasculature. Panel a shows: Healthy condition. Left: A circulating leukocyte (open blue arrowhead); single frame from a video; and Right: A rolling leukocyte (blue arrowhead); averaged image of five single (consecutive) frames from another video. Panel b shows: Inflamed condition. Left: A slow-rolling leukocyte (red arrowhead); and Right: Slow-rolling and adherent leukocytes (red and black arrowheads, respectively) from another video. Panels c-d show: Healthy and inflamed oral mucosa tissue. Panels e-g show: Velocity and displacement profiles of individual leukocytes. In Panel “e”, we have used unpaired t-test (blue column, n=18; Red column, n=39). P-value<0.0001. *Note: Images of the inflamed tissue were obtained with one version of the instrument (see FIG. 26). Data acquisition parameters and image processing pipelines are detailed in Example 1.

FIG. 28 shows data analyses of the region of interest (ROI). This includes a representative frame image from a 2884-frame video, recorded at 300 fps (see Panel a), intensity profile in the ROI #1 (Panel b), and intensity profile in the ROI #2 (Panel c).

FIG. 29 shows the ratio between ROI #2 and ROI #1 which reduces background and allows extraction of the intensity peaks that represent the white blood cell candidates.

FIG. 30 shows images of the oral mucosa's microvasculature and its content. Circulating white blood cells are indicated with red arrows. Dark cells are the red blood cells. Panels a-c show granulocyte candidate and Panels d-e show lymphocyte candidate.

FIG. 31 shows images of the oral mucosa's microvasculature and its content. Rolling white blood cells are indicated with arrows. The images show a “T” on the top left, which stand for the time.

FIG. 32 shows images of the oral mucosa's microvasculature and its content. Panel a shows canker sores (also known as aphthous ulcers), which are indicated with arrows. Panel b shows an example of white blood cell recruitment during local infection/inflammation.

FIG. 33 is a flow chart depicting the image data processing pipeline.

FIG. 34 shows Region-of-interest selection. Panel a. Original image. Panel b. Initial, automated “guess” of the ROI area by time projected standard deviation and segmentation; vessel lumen pixels in which movement is recorded appear bright. Panel c. User-specified confirmation of foreground (blue) and background (red) pixels for graph cut segmentation. Panel d. User-specified line eraser correction applied to fix graph cut segmentation errors. Here, the error is caused by the segmentation algorithm interpreting the vessel loop to be closed. The line eraser is used to separate the actual “overpass” and “underpass” stretches of the vessel in the loop. Panel e. Segmentation of the vessel, post-correction (red) and its skeleton (dark line in segmented region). Panel f. User-specified polygon (teal), including only the in-focus region of the vessel. The ROI is designated by the pixels that are both within the vascular area (orange) and within the user-specified polygon (teal).

FIG. 35 shows an example of a transformed coordinate system, the skeleton coordinate system (SCS). Panel a. The two axes of SCS run parallel and normal to the ROI's blood flow respectively. Panels b-f. How SCS is generated. Panel b. The ROI's binary mask is skeletonized. The skeleton runs along the ROI's axis, parallel to blood flow. Panel c. The skeleton is “duplicated” with equidistanced separation using image dilation followed by edge finding. Panel d. Enough skeleton duplicates are generated to span the full ROI width; they form the y′-gridlines of the SCS. Panel e. The normal line of a skeleton pixel has a slope perpendicular to the skeleton's slope at the pixel, and intersects the pixel. Panel f. Normal lines drawn at all skeleton pixels define the x′ gridlines of the SCS. Panel g. The SCS is versatile towards different vessel morphologies, including curved ROIs. Panel h. SCS grid points (green) overlaid on a sinuous vessel. Different shades of green denote different vessel blocks, which divide the ROI into units along the x′ axis suitable for perfusion parameter measurements. Panel i. SCS grid points (green) overlaid a strongly curved vessel.

FIG. 36 shows blood flow velocity measurement using space-time diagrams. Panel a. Velocity is proportional to the gradient of the sloped lines in these diagrams. Since velocity is a function of x′ and time, the space-time diagrams are divided into vessel blocks (different shades of green) and time segments (orange). Each diagram corresponds to one y′-gridline. Panels b-e. Blood flow velocity calculation. Panel b. Space-time diagram of one y′-gridline, vessel block and time segment. Panel c. Mask applied to the diagram in Fourier space. Noise and large objects (including large leukocytes) are excluded, as the latter may travel more slowly than red blood cells. Panel d. Space-time diagram after masking. Panel e. Segmentation of (d.) creates a binarized image of lines, the gradient of which can be measured. The velocity value for the given space-time diagram is calculated from the weighted average of these gradients, with the weights (intensity of each line) determined by the line's aspect ratio. Panel f. The final reported velocity values, as a function of time and vessel block. Each value may be the average of velocities from one or more skeletons. Only the skeletons within 50% of the minimum ROI width are considered. Each time point, defined as the midpoint of a time segment, averages the values from the multiple time segments that overlap it (a.).

FIG. 37 shows counting and locating candidate leukocytes. Panel a. Space-time diagram of one y′-gridline, vessel block and time segment. Each bright band suggests presence of a candidate leukocyte. Panel b. The diagram is rotated according to the gradient angle determined during blood flow velocity measurement, and a moving sum is performed along the horizontal direction. The outcome is an intensity profile with peaks corresponding to the candidate leukocytes crossing the given y′-gridline in the given vessel block, during the given time segment. Panels c-f. Consolidation of intensity profiles from all space-time diagrams. Panel c. Consolidation along y′ axis, by y′-gridlines. Intensity profiles from different y′-gridlines (index sⱼ) at each vessel block and time segment are sorted pixel-by-pixel and ranked. The outcome are vectors of the same pixel count as the intensity profiles, with m₁ containing the maximum intensity value of all y′-gridlines at each of its pixels, m₂, the 2nd highest intensity value of all y′-gridlines, etc. Only the first nₛ of these mⱼ vectors are kept, with nₛ equal to the number of y′-gridlines needed to span the smallest leukocyte size. The values of these vectors are then averaged, pixel by pixel, to yield the final y′-consolidated intensity profile. Panel d. Consolidation along t axis, by time segments. y′-consolidated intensity profiles are pieced together along the time axis for each vessel block. The four peaks in (b.) are indicated in blue numbers, and the time segment of the space-time diagram in (a.) is indicated by the orange box. Panel e. y′t-consolidated intensity plots of two vessel blocks show the time delay of candidate leukocytes travelling from upstream (Blk01) to downstream (Blk02). The time delay shows slight variations from cell to cell. Consolidation along x′ axis, by vessel blocks, must first align the peaks, match them in position. Panel f. Consolidation along x′ axis, by vessel blocks. Outcome of peak matching between two vessel blocks by dynamic time warping (DTW). After peak matching, the two intensity plots are summed. A threshold is defined and each peak exceeding the threshold is analyzed. The peak position and width correspond to the time of appearance of the candidate leukocyte in the video. As DTW warps the time axis for each matched vector, “un-warping” based on the warping function for each vessel block is necessary, and the resulting peak widths may not be the same between vessel blocks. Panel g. Peak information from (f.) is transferred to the space-time diagram shown in (a.). The red vertical lines indicate the peak position and width for each vessel block. The gradient is determined from the pixel shift required for DTW peak matching. Panel h. A “gate” (red) is drawn for each candidate leukocyte based on the peak widths and gradient. The horizontal width of these gates will approximate the spatial location of appearance of the cell (see Panel j.) Panel i. Each gate is indexed, and the total number of gates in the video are counted and reported as the candidate leukocyte count estimate. Panel j. Gated pixels for each gate index correspond to the approximate volume in the video in which a candidate leukocyte appears. Drawing a boundary around the gated pixels in one time frame (orange dotted line) of each of the four gates yields a bounding box (yellow) in each frame restricting spatially the likely location of a candidate leukocyte. Coordinates of these bounding boxes will determine where input data will be cropped for deep learning leukocyte detection.

Like reference numerals will be used to refer to like parts from Figure to Figure in the following description of the drawings.

### DETAILED DESCRIPTION OF THE INVENTION

Before the present invention is described in further detail, it is to be understood that the invention is not limited to the particular embodiments described. It is also to be understood that the terminology used herein is for the purpose of describing particular embodiments only and is not intended to be limiting. The scope of the present invention will be limited only by the claims. As used herein, the singular forms “a”, “an”, and “the” include plural embodiments unless the context clearly dictates otherwise.

It will be appreciated by those skilled in the art that while the disclosed subject matter is described herein in connection with particular embodiments and examples, the invention is not necessarily so limited, and that numerous other embodiments, examples, uses, modifications and departures from the embodiments, examples and uses are intended to be encompassed by the claims attached hereto.

It should be apparent to those skilled in the art that many additional modifications beside those already described are possible without departing from the inventive concepts. In interpreting this disclosure, all terms should be interpreted in the broadest possible manner consistent with the context. Variations of the term “comprising”, “including”, or “having” should be interpreted as referring to elements, components, or steps in a non-exclusive manner, so the referenced elements, components, or steps may be combined with other elements, components, or steps that are not expressly referenced. Embodiments referenced as “comprising”, “including”, or “having” certain elements are also contemplated as “consisting essentially of” and “consisting of” those elements, unless the context clearly dictates otherwise.

The present invention provides a system that enables stabilized noninvasive imaging of the oral mucosa microvasculature with high spatial resolution. The system may have two key components: (1) an oral mucosa stabilizer and (2) a miniaturized imaging instrument. The two components together enable label-free, high-resolution imaging of the exposed microvasculature and its content (red and white blood cells, platelets, endothelial tissue/cells, epithelium, etc.) while minimizing motion artifacts. The obtained imaging data is processed using a custom algorithm adapted to the system. The results (e.g., blood cell count estimate, motion and subtype classification, etc.) provide valuable medical information about the patient's immune system. The system may be used as a diagnostic tool for safe daily monitoring of medical conditions of healthy (preventive diagnosis) and unhealthy patients (e.g., acute and systemic inflammation, sepsis, tissue hypoxia, etc.). While being part of the same imaging system for noninvasive imaging of the oral mucosa's microvasculature, each of the key components are often presented separately below. The different oral mucosa sections can include, but are not limited to: the buccal mucosa, the lips (external and internal sections), the floor of the mouth, the tongue, the gingiva, the palates, the tonsils, etc.

An embodiment of the oral mucosa stabilizer can include the following features:
mechanical holder(s) with the capability to modify the natural position of the oral mucosa tissue by means of mechanical contact;
mechanical holder(s) with the capability to maintain the modified position of oral mucosa tissue;
mechanical holder(s) that expose the microvasculature of the oral mucosa tissue;
mechanical holder(s) that enable privileged access to the oral mucosa's microvasculature for a data acquisition instrument(s);
mechanical holder(s) that modify the natural position of the oral mucosa tissue with or without integrated optics (e.g., lenses) or light sources (e.g., LEDs); and
combination of mechanical holders that maintain the modified position of the oral mucosa tissue.

An embodiment of the oral mucosa stabilizer can serve any number of the following purposes:
modify the natural position of the oral mucosa tissue in a noninvasive manner;
expose the oral mucosa's microvasculature;
enable privileged access for a data acquisition instrument;
maintain the stabilized position of the oral mucosa's tissue during data acquisition; and
minimize the mechanical vibrations of the oral mucosa tissue during data acquisition.

Data acquisition instruments include, but are not limited to the following optical techniques: single-photon, multi-photon, confocal, laser scanning, system with a coherent light source, system with a non-coherent light source (e.g., LED, halogen lamps), etc.

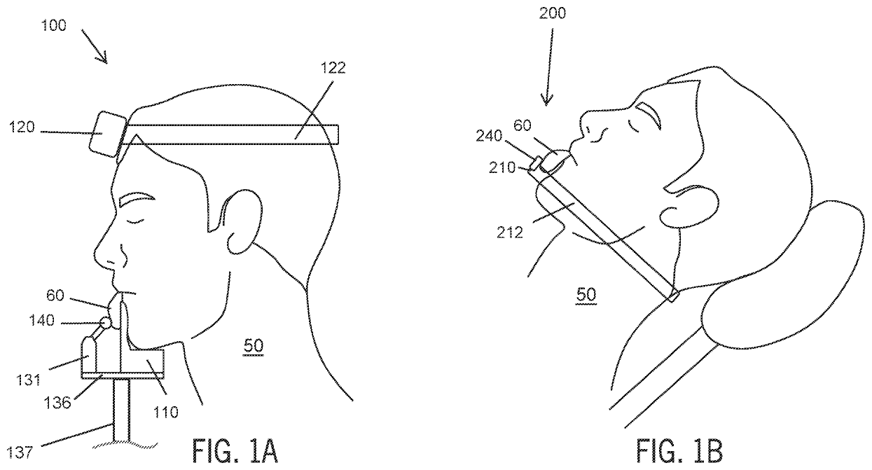

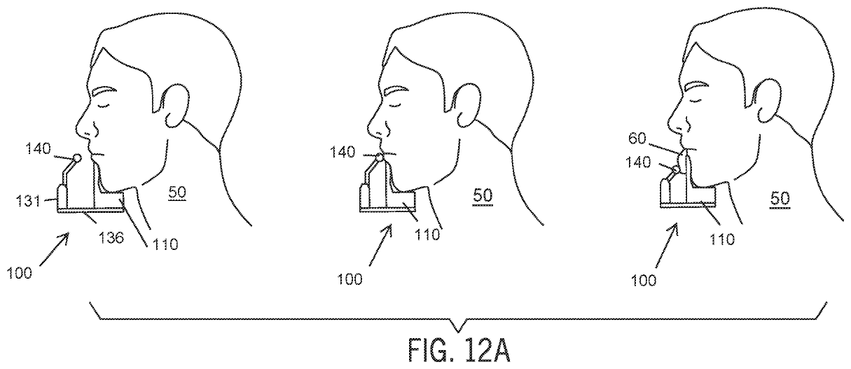

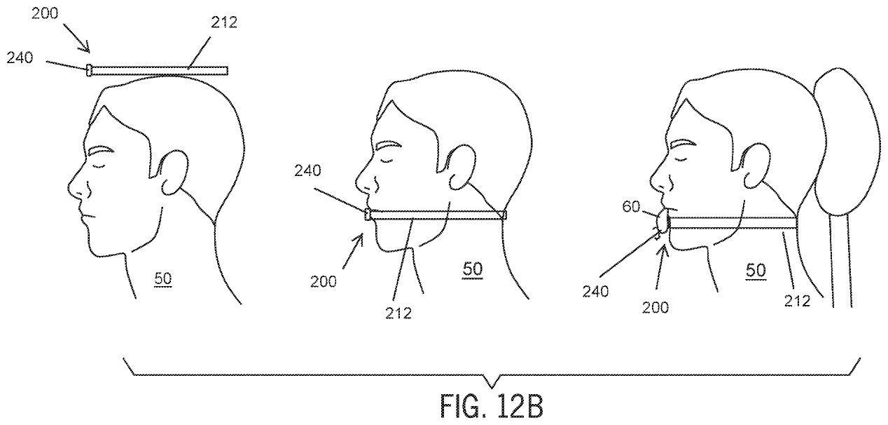

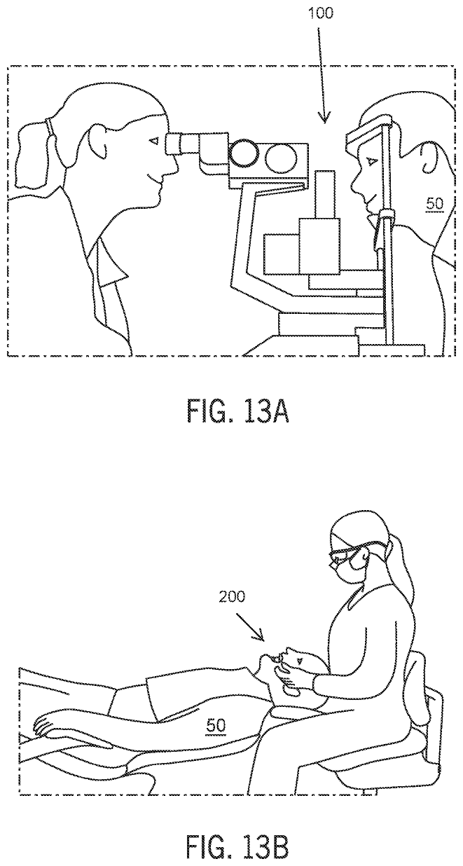

Turning to FIG. 1A, there is shown one example embodiment of a fixed bench-top tissue stabilizer 100 according to the invention positioned on a human subject 50. The bench-top tissue stabilizer 100 includes a chin holder 110, and a forehead holder 120 secured to the subject 50 by a strap 122. The chin holder 110 includes a vertical sliding bar 131 and a horizontal sliding bar 136 on a base of the chin holder 110 for adjusting vertical and horizontal position of an adapter 140 which modifies the natural position of the oral mucosa tissue 60 in a noninvasive manner and maintains the stabilized position of the oral mucosa's tissue 60 during data acquisition. A vertical bracket rod 137 connects the bench-top tissue stabilizer 100 to a bench top. FIG. 12A shows a method of using of the bench-top tissue stabilizer 100 where the adapter 140 is moved toward the subject 50 (left), placed in contact with the subject (center), and then the adapter 140 maintains the stabilized position of the oral mucosa's tissue 60 (right). FIG. 13A shows a method of using of the bench-top tissue stabilizer 100 in a system for imaging of microvasculature of tissue of a subject 50 according to the invention in which the stabilizer can be used with the subject sitting.

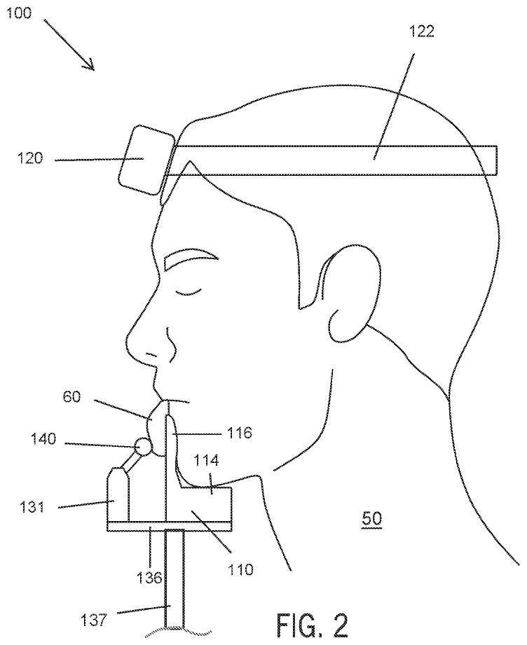

Referring now to FIGS. 1B and 2, there is shown one example embodiment of a portable tissue stabilizer 200 according to the invention positioned on a human subject 50. The portable tissue stabilizer 200 includes a holder 210 for adjusting for an adapter 240 which modifies the natural position of the oral mucosa tissue 60 in a noninvasive manner and maintains the stabilized position of the oral mucosa's tissue 60 during data acquisition. An elastic strap 212 keeps the holder 210 secured to the subject 50. The tissue stabilizer 200 is portable and can be carried by the subject (adult, infants, neonates). FIG. 12B shows a method of using of the portable tissue stabilizer 200 where the adapter 240 is moved downward toward the subject 50 (left), placed in contact with the subject (center), and then the adapter 240 maintains the stabilized position of the oral mucosa's tissue 60 (right). FIG. 13B shows a method of using of the portable tissue stabilizer 200 on a human subject 50 in which the stabilizer can be used with the subject lying down.

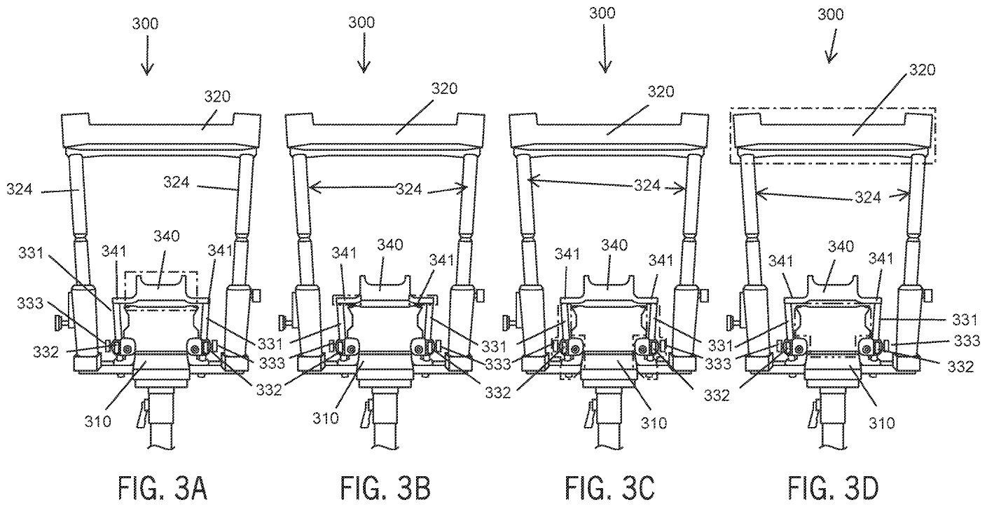

Looking now at FIGS. 3A-3B, there is shown one example embodiment of a multicomponent stabilizer tissue stabilizer 300 according to the invention. In FIG. 3A, an adapter 340 is noted by a dashed rectangle. In FIG. 3B, adapter holders 341 are noted by dashed rectangles. In FIG. 3C, a sliding mechanism is noted by dashed rectangles. In FIG. 3D, a chin holder 310 and a forehead holder 320 are noted by dashed rectangles, respectively. The tissue stabilizer 300 includes a chin holder 310 and a forehead holder 320 mounted on a frame 324. The sliding mechanism adjusts vertical position of the adapter 340, and includes vertical sliding bars 331 on which are mounted movable collars 332 that may be fixed in place by fixation screws 333. The adapter 340 is mounted to the vertical sliding bars 331 by adapter holders 341. The vertical sliding bars 331 may be moved up and down and then fixed in place by the fixation screws 333 to adjust vertical position of the adapter 340 to modify the natural position of the oral mucosa tissue 60 in a noninvasive manner and maintain the stabilized position of the oral mucosa's tissue 60 during data acquisition.

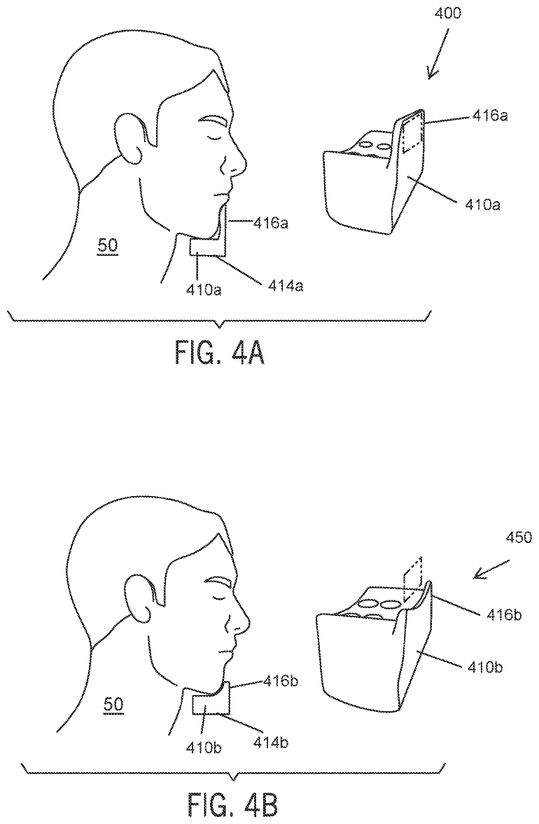

Turning to FIG. 4A, there is shown one example embodiment of a chin holder 400 of a tissue stabilizer according to the invention positioned on a human subject 50. The chin holder 400 includes a base 410a with a horizontal wall 414a connected to a vertical wall 416a. The lip's oral mucosa tissue 60 is unrolled and positioned against the vertical wall 416a of the chin holder 400. To stabilize the imaging area, the oral mucosa tissue 60 can be pushed (gently) against a solid surface of the vertical wall 416a of the chin holder 400.

Referring now to FIG. 4B, there is shown one example embodiment of a chin holder 450 of a tissue stabilizer according to the invention positioned on a human subject 50. The chin holder 450 includes a base 410b with a horizontal wall 414b connected to a vertical wall 416b. The oral mucosa tissue 60 is pushed against the human subject.

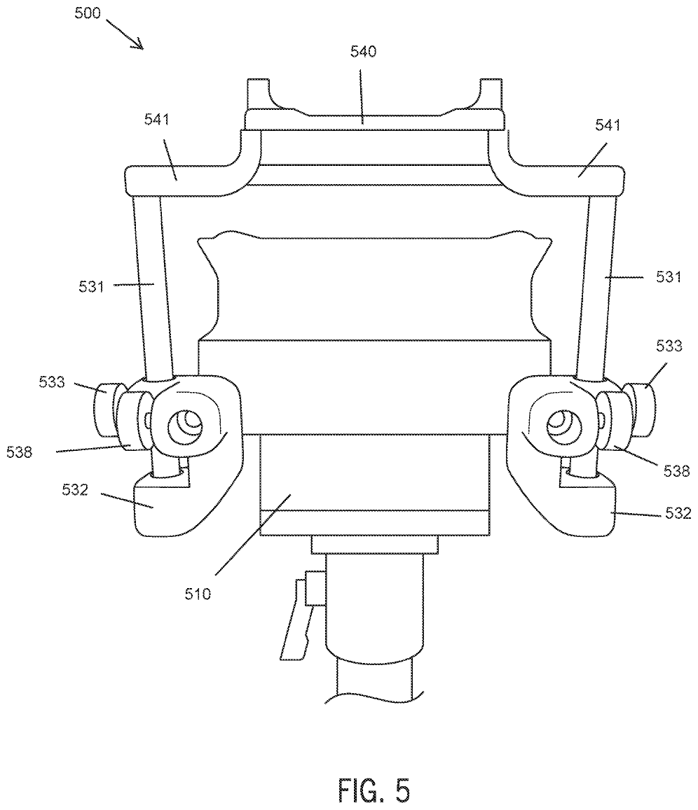

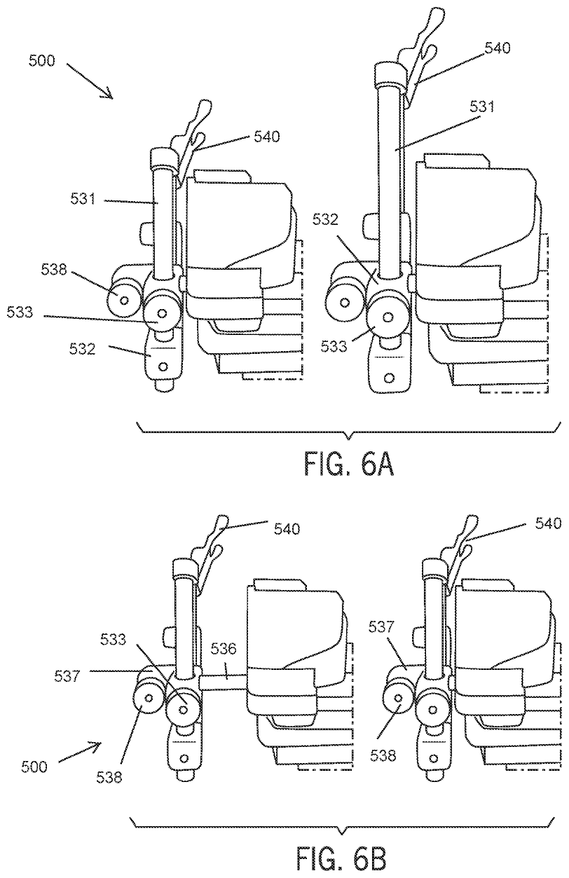

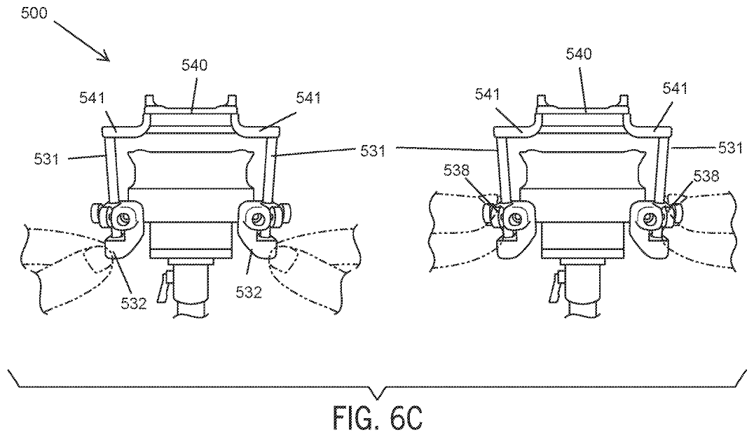

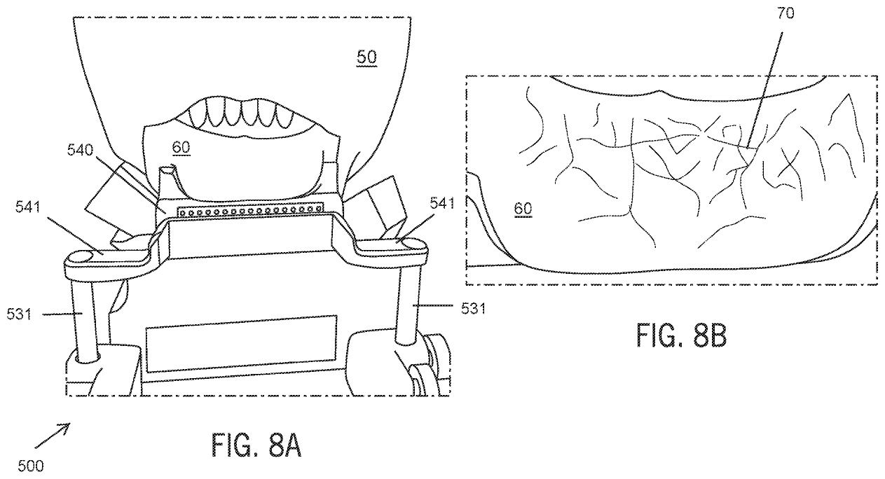

Looking now at FIGS. 5, 6A, 6B and 6C, there is shown is one example embodiment of a multicomponent tissue stabilizer 500 according to the invention. FIGS. 8A and 8B show the multicomponent stabilizer tissue stabilizer 500 during use with a human subject 50. FIG. 8B is a detailed view of the oral mucosa tissue area, in which the microvasculature 70 is exposed for investigation and imaging. A sliding mechanism provides for precise positioning of the stabilizer and optimization of the pressure applied on the oral mucosa tissue 60. The tissue stabilizer 500 includes a chin holder 510. A sliding mechanism adjusts vertical position of the adapter 540, and includes vertical sliding bars 531 on which are mounted movable collars 532 that may be fixed in place by fixation screws 533. The adapter 540 is mounted to the vertical sliding bars 531 by adapter holders 541. The vertical sliding bars 531 may be moved up and down and then fixed in place by the fixation screws 533 to adjust vertical position of the adapter 540 to modify the natural position of the oral mucosa tissue 60 in a noninvasive manner and maintain the stabilized position of the oral mucosa's tissue 60 during data acquisition. The sliding mechanism also includes horizontal sliding bars 536 on which are mounted movable collars 537 that may be fixed in place by fixation screws 538. The horizontal sliding bars 538 may be moved laterally and then fixed in place by the fixation screws 538 to adjust horizontal position of the adapter 540 to modify the natural position of the oral mucosa tissue 60 in a noninvasive manner and maintain the stabilized position of the oral mucosa's tissue 60 during data acquisition.

Thus, a sliding system (e.g., FIGS. 3A-3B, 5, 6) enables the spatial positioning (linear or angular) of the adapter of the tissue stabilizers. This produces a normal force to maintain the modified position of the tissue, and allows adjustment and optimization of the normal force applied on the tissue. It contributes to the stabilization of the tissue, and enables rapid adjustment and fixation of the tissue stabilizers (within 0-10 minutes) prior to and during the imaging. The operation is typically manual, but the system can be automated.

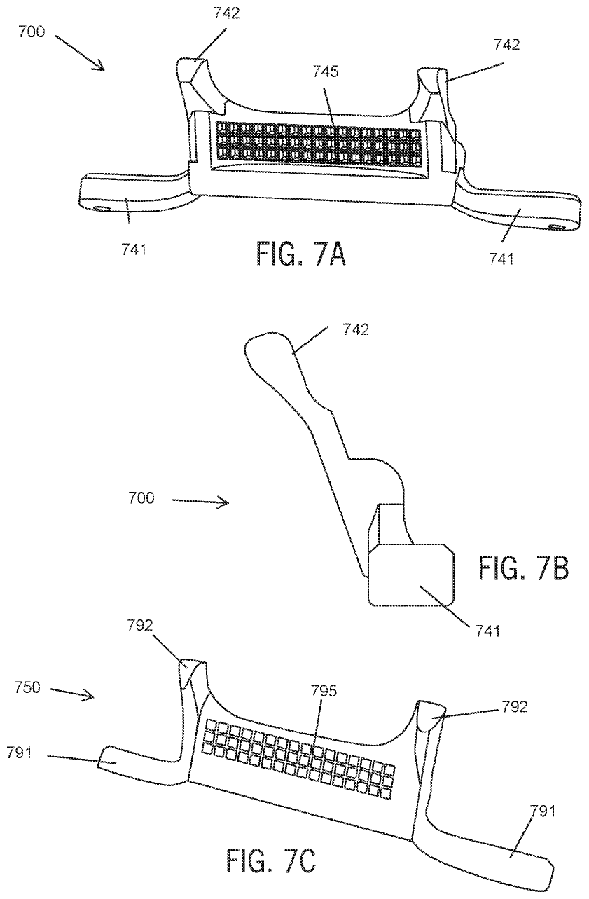

Turning to FIGS. 7A and 7B, there is shown one example embodiment of an adapter 700 of a tissue stabilizer according to the invention. The adapter 700 includes adapter holders 741 for mounting to a sliding mechanism as in the tissue stabilizer 500. Opposed tabs 742 create side pressure points that enhance the stabilization of the oral mucosa tissue 60. This contributes to the minimization of undesired vibrations and movement artifacts, and flattens the surface of the exposed tissue area. The tabs 742 help maintain the focus (in depth) during the navigation (mechanical and optical) across the tissue. This contributes to the optimization of tissue position and the exposure of the microvasculature prior to and during data acquisition. The lateral (XY) dimensions of pressure points usually vary between about 0.1-5 mm.

FIG. 7B shows adapted curvature which suits the surface curvature of the oral mucosa tissue. This contributes to the uniform distribution of the mechanical pressure applied on the oral mucosa tissue 60, and minimizes the discomfort of the subject. This contributes to the optimization of tissue position and the exposure of the microvasculature prior to and during the data acquisition. The radius of curvature usually varies between about 2-100 mm. Still referring to FIG. 7B, the tilted design suits the surface curvature of the oral mucosa tissue, and contributes to the uniform distribution of mechanical pressure applied on the oral mucosa tissue. This minimizes the discomfort of the subject, and optimizes the position of the tissue and exposes the microvasculature during data acquisition. The tilt angle usually varies between about 45-90 degrees.

Looking at FIG. 7A, a patterned surface finish 745 of the adapter 700 increases the contact area with the oral mucosa tissue 60, and increases the friction between the oral mucosa tissue and the stabilizers. This contributes to the stabilization of the oral mucosa tissue, and optimizes the position of the tissue and exposes the microvasculature during data acquisition. The spacing between a repeated feature (e.g., extruded circle, square, etc.) usually varies between about 1-15 mm. The dimensions of the repeated feature usually vary between about 1-5 mm. in each dimension (X, Y, Z). A vacuum created by the adapter 700 generates air suction to maintain the position of the oral mucosa tissue. This optimizes the position of the tissue and exposes the microvasculature during data acquisition. The vacuum pressure varies between about 0.1-5 SCFH.

Turning to FIG. 7C, there is shown another example embodiment of an adapter 750 of a tissue stabilizer according to the invention. The adapter 750 includes adapter holders 791 for mounting to a sliding mechanism as in the tissue stabilizer 500. Opposed tabs 792 create side pressure points that enhance the stabilization of the oral mucosa tissue. The adapter 750 has a patterned surface finish 795 that functions similarly to the patterned surface finish 745 in adapter 700.

Various dimensions are possible for the adapters 700, 750. For section(s) in contact with the oral mucosa area, dimensions can be: X: 1-100 mm.; Y: 1-100 mm.; and Z: 1-100 mm. Tabs 742, 792 can create side pressure points with dimensions such as: X: 1-50 mm.; Y: 1-50 mm.; and Z: 1-50 mm. For the vacuum area, dimensions can be: X: 1-100 mm.; Y: 1-100 mm.; and Z: 1-100 mm. For the patterned texture area 745, dimensions can be: X: 1-100 mm.; Y: 1-100 mm.; and Z: 1-100 mm. For steps of the patterned texture area 745, dimensions can be: X: 0.1-20 mm.; Y: 0.1-20 mm.; and Z: 0.1-20 mm.

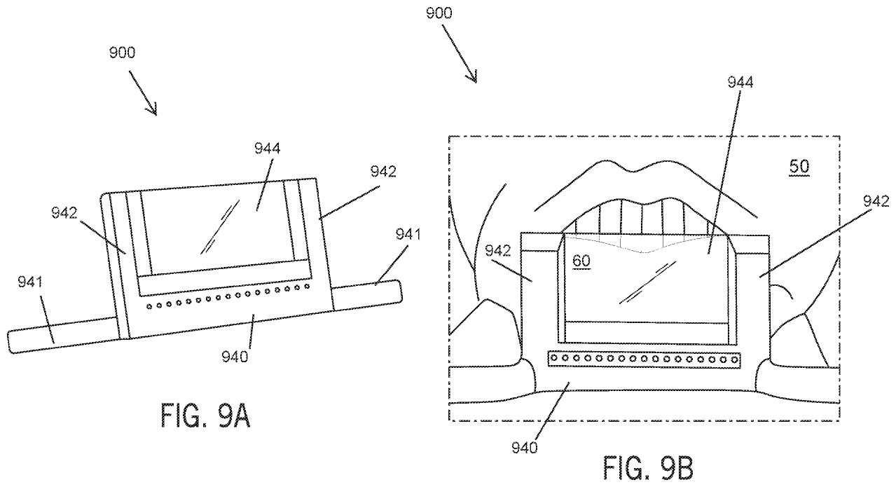

Referring now to FIGS. 9A and 9B, there is shown another example embodiment of an adapter 900 of a tissue stabilizer according to the invention. The adapter 900 includes a lower wall 940 and adapter holders 941 for mounting to a sliding mechanism as in the tissue stabilizer 500. Opposed tabs 942 create side pressure points that enhance the stabilization of the oral mucosa tissue 60. This contributes to the minimization of undesired vibrations and movement artifacts, and flattens the surface of the exposed tissue area. The tabs 942 help maintain the focus (in depth) during the navigation (mechanical and optical) across the tissue. This contributes to the optimization of tissue position and the exposure of the microvasculature prior to and during data acquisition. A transparent sheet 944 is mounted between the tabs 942. Materials of the adapter 900 allow for optimization of the performance of the stabilizer (e.g., glass sheet 944+biocompatible photo-resin). The number of materials usually varies between about 1-5. A coating can modify the surface finish texture to enhance the friction contact, to make the material biocompatible (sterilizable), and make the material hydrophobic (e.g., SigmaCote® siliconizing reagent), etc.

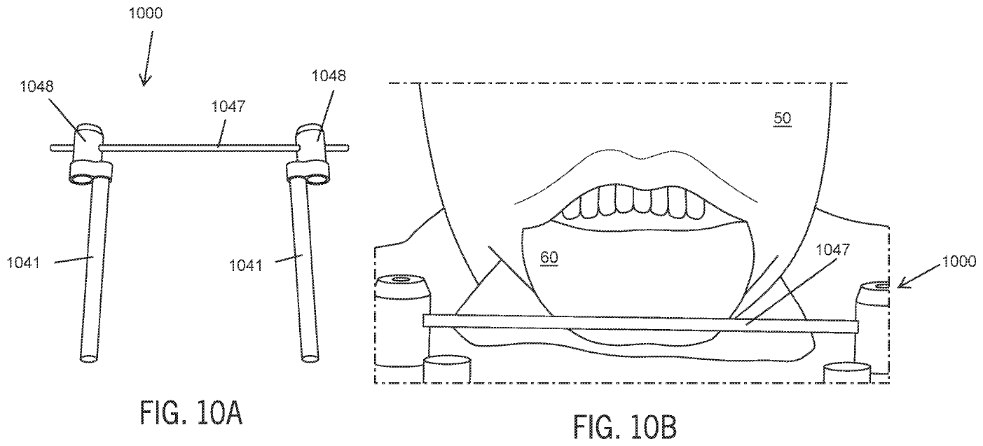

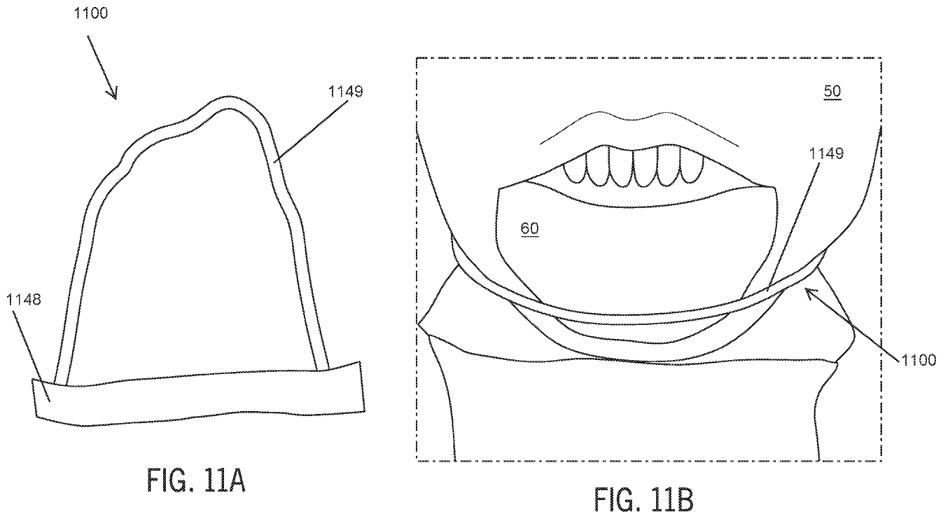

Looking now at FIGS. 10A and 10B, there is shown another example embodiment of an adapter 1000 of a tissue stabilizer according to the invention. The adapter 1000 includes adapter holders 1041 for mounting to a sliding mechanism as in the tissue stabilizer 500. A rod 1047 is connected to the adapter holders 1041 using connectors 1048. FIG. 10B shows the adapter 1000 during use with a human subject 50, in which the microvasculature is exposed for future investigation and imaging and the rod 1047 stabilizes the oral mucosa tissue 60 for imaging.

Turning to FIGS. 11A and 11B, there is shown another example embodiment of an adapter 1100 of a tissue stabilizer according to the invention. The adapter 1100 includes an adapter holder 1148 for mounting to a sliding mechanism as in the tissue stabilizer 500. A flexible elastic loop 1149 is connected to the adapter holder 1148. FIG. 11B shows the adapter 1100 during use with a human subject 50, in which the microvasculature is exposed for future investigation and imaging and the loop 1149 stabilizes the oral mucosa tissue 60 for imaging.

One method of using a tissue stabilizer according to the invention includes the steps of:
Step 1. Position the subject (see FIGS. 12A (left), 12B (left)).
Step 2. Adjust the position of the stabilizer next to the oral mucosa's tissue of interest (see FIGS. 12A (center), 12B (center)).
Step 3. Unroll the oral mucosa's tissue of interest to expose the microvasculature (see FIGS. 12A (right), 12B (right)).

Fine positioning can be achieved to maintain the optimized position of oral mucosa tissue as the stabilizer is applying a mechanical force on the non-imaging area of the tissue. The amplitude of the force is optimized to avoid blood flow alternations in the microvasculature of the oral mucosa tissue. Parameters to adjust to maintain the position of the oral mucosa tissue without altering the blood flow include:
Spatial positioning of the stabilizer with respect to the oral mucosa tissue (e.g., FIGS. 5, 6A-6C).
Intensity of the vacuum suction system of the stabilizer.
Material(s) of the stabilizer.
Mechanical design of the stabilizer (the geometrical shape, curvatures) (FIGS. 3A-3D).
Surface finish/texture of the area of the stabilizers that is in contact with the oral mucosa tissue (e.g., the patterned surface, coating, etc.) (FIG. 7A-7C).

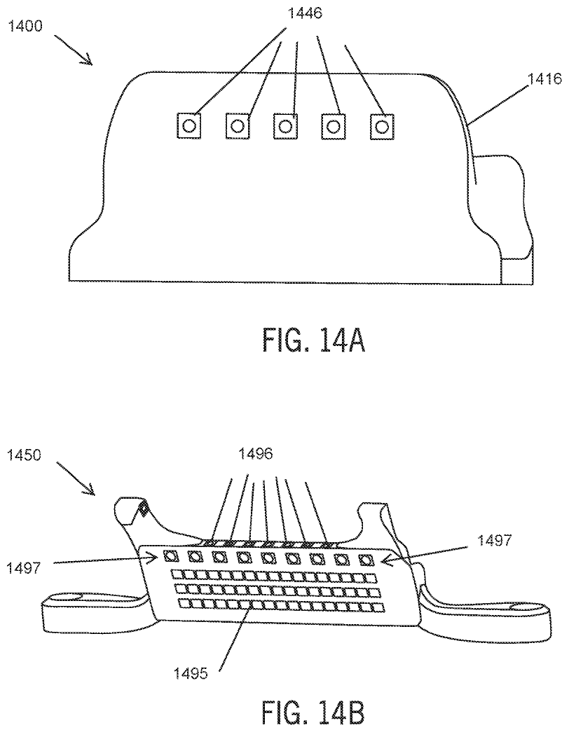

Referring now to FIG. 14A, there is shown another example embodiment of a chin holder 1400 of a tissue stabilizer according to the invention. The chin holder 1400 includes a vertical wall 1416 with light-outputting ends 1446 of optical fibers transmitting light from light sources of an illumination device. Integrated light source and optics enables the illumination (oblique back-illumination, offset trans-illumination, etc.) of the oral mucosa tissue, and enables the visual examination of the tissue, and enables illumination for a data acquisition instrument(s).

Looking now at FIG. 14B, there is shown another example embodiment of an adapter 1450 of a tissue stabilizer according to the invention. The adapter 1450 includes a patterned surface finish 1495 that increases the contact area with the oral mucosa tissue. The adapter 1450 includes light-outputting ends 1496 of optical fibers transmitting light from light sources of an illumination device. The adapter 1450 includes a line of objective lenses 1497 collecting scattered light from the tissue 60. Integrated light source and optics enables the illumination (oblique back-illumination, offset trans-illumination, etc.) of the oral mucosa tissue, and enables the visual examination of the tissue, and enables illumination for a data acquisition instrument(s).

Thus, integrated light source and optics (e.g., FIGS. 14A and 14B) enables the illumination (oblique back-illumination, offset trans-illumination, etc.) of the oral mucosa tissue. It enables the visual examination of the tissue, and enables illumination for a data acquisition instrument(s).

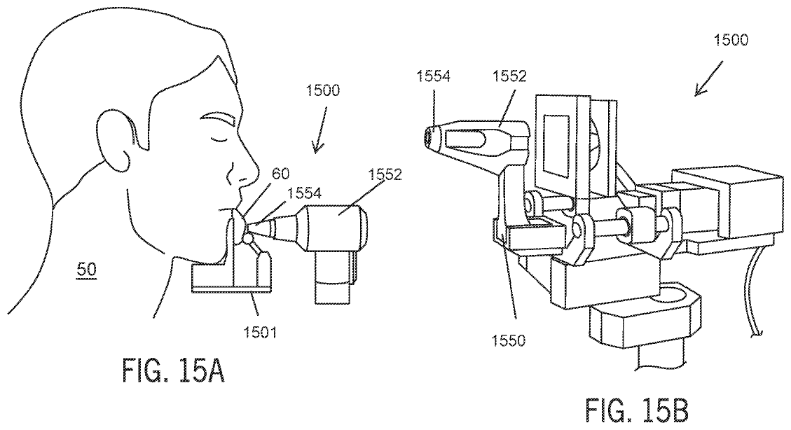

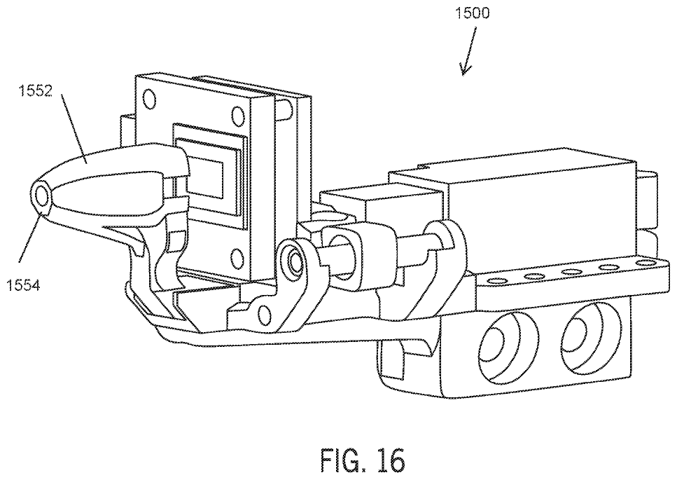

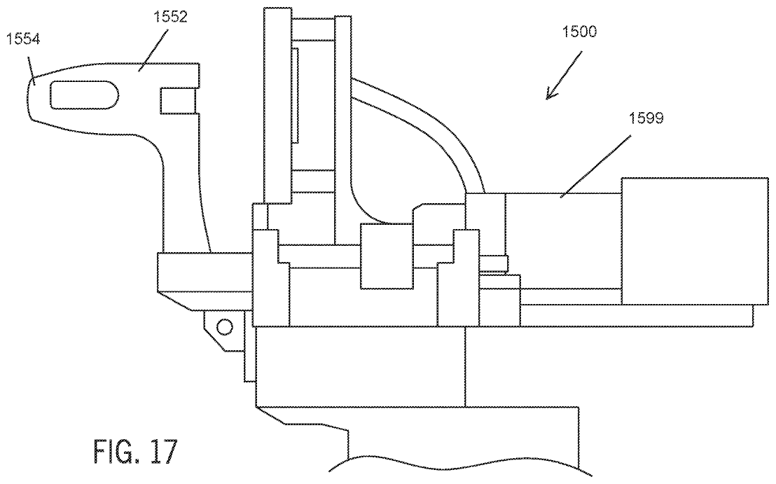

Turning to FIGS. 15A, 15B, 16 and 17, there is shown components of an example embodiment of a system 1500 according to the invention having a bench-top tissue stabilizer 1501 positioned on a human subject 50 and an imaging instrument 1550. The imaging instrument 1550 includes a housing 1552 and an imaging section with a tip 1554. The housing may be miniaturized (e.g., about 5 cm. long). The bench-top tissue stabilizer 1501 may be structured as tissue stabilizer 100 described above. Focus adjustment can use at least two methods:
1. Method 1: Mechanical displacement of the camera or of the relay optics using an actuator motor 1599 (see FIG. 17). This includes a sliding system to maintain the precise alignment of the moving element (lens or camera/sensor); or
2. Method 2: Electric focus adjustment using electrically tunable lenses (e.g., liquid crystal lenses, electrowetting lenses, etc.).

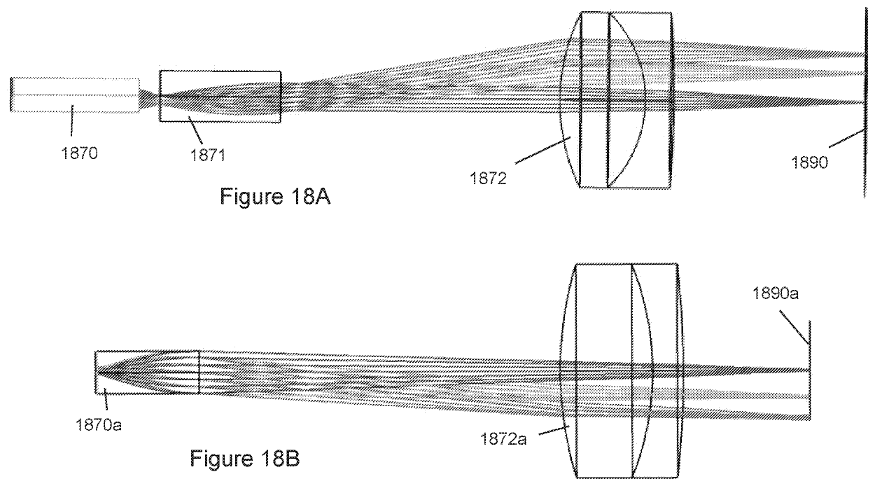

FIG. 18A is an optical design of a light collection pathway of an example embodiment of an imaging instrument of a system according to the invention. The optical design includes an aberration-corrected gradient index (GRIN) lens objective (NA≈0.75) 1870, a relay GRIN lens 1871, a double achromat lens 1872, and an image detector (e.g., sensor/camera) 1890. Among the advantageous features of one embodiment of the imaging instrument are a high-speed CMOS sensor (up to 1000 fps) and a miniaturized aberration corrected GRIN lens objective (NA≈0.75) for high-resolution monitoring of rapidly moving leukocytes in the blood circulation.

FIG. 18B is another optical design of a light collection pathway of another example embodiment of an imaging instrument of a system according to the invention. The optical design includes a standard imaging GRIN lens objective (NA≈0.5) 1870a, a double achromat lens 1872a, and an image detector (e.g., sensor/camera) 1890a.

A feature of the optical designs of FIGS. 18A and 18B is a light collection pathway having three main components:
1. Objective Lens. Lenses collecting the light from the tissue, particularly using specific objective lenses called Gradient Index (GRIN) lenses. GRIN lenses are small (diameter about 0.5-5 mm., length about 0.5-10 mm.) and have very short working distances (0-200 μm) that are well adapted for the present application that targets the shallow microvasculature. GRIN lenses also have a high Numerical Aperture (NA about 0.5-1) that increases the optical resolution of systems of the invention. (see FIG. 18A). Moreover, a system of the invention can use a very specific GRIN lens assembly that is aberration-corrected, has a high NA of about 0.75, and generates a magnification of about 3-5 by itself.
2. Relay optics. The relay optics transport light from the objective lens to the sensor. An aberration corrected (e.g., double achromat lens) is advantageous. The relay optics can be single or multiple lenses (see FIGS. 18A and 18B).
3. Sensor/Camera. When used as the image detector, the sensor/camera can be monochrome (white/black), binning 2×2 or 4×4, visible and near infra-red wavelengths, high acquisition rates (from 30-1000 fps), and have a compact size. See FIGS. 18A and 18B.

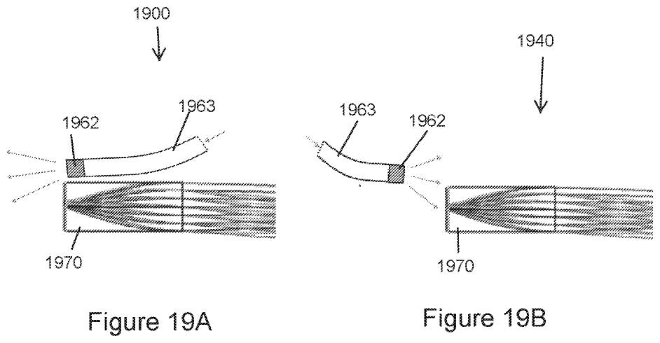

FIG. 19A shows an example light injection configuration and imaging modality of an imaging instrument 1900 which includes a GRIN lens objective 1970 and includes a light-outputting end 1962 of an optical fiber 1963 transmitting light from a light source. The illumination device uses Oblique Back-Illumination (OBM).

FIG. 19B shows another example light injection configuration and imaging modality of an imaging instrument 1940 which includes a GRIN lens objective 1970 and includes a light-outputting end 1962 of an optical fiber 1963 transmitting light from a light source. The illumination device uses Offset Trans-Illumination (OTM).

Thus, the position of the illumination source (e.g., optical fiber) with respect to the light collection optics defines the imaging modality. Three different non-limiting imaging modalities can be considered with the illumination source in three different positions: (1) Oblique Back-Illumination (OBM) (see FIG. 19A), and (2) Offset Trans-Illumination (OTM) (see FIG. 19B).

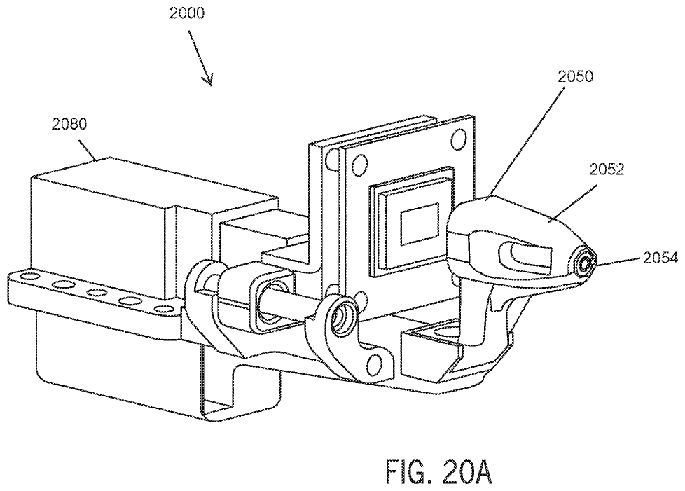

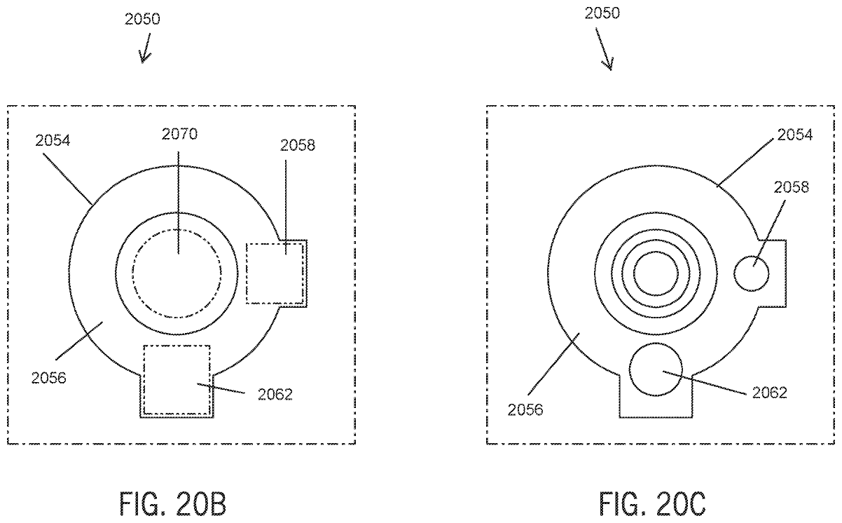

Referring now to FIGS. 20A, 20B, and 20C, there is shown another example embodiment of an imaging instrument 2050 of a system 2000 according to the invention. The imaging instrument 2050 includes a housing 2052 and an imaging section with a tip 2054. The system includes a controller 2080 which may include a memory and an electronic processor coupled to the memory. The tip 2054 has an annular vacuum channel 2056, an irrigation channel 2058, a light-outputting end 2062 of an optical fiber transmitting light from a light source, and an objective lens 2070.

The imaging tip 2054 has advantageous characteristics. For example, the gradient index lens (objective lens 2070) has a cavity that stabilizes the GRIN lens objective. Particularly, the diameter of the cavity is slightly smaller (10-100 μm) than the diameter of the GRIN lens. The insertion of GRIN objective lens 2070 induces expansion of the cavity and a force normal to the surface of the lens. This normal force generates friction that maintains the GRIN objective lens 2070 in the cavity without using any additional mechanical support (e.g., screws). An Illumination source cavity allows insertion of the illumination source (fiber or LED) next to the objective lens. The vacuum channel 2056 allows stabilization of the tissue using air suction. Particularly, the diameter of the vacuum channel 2056 is small and compact for a specific purpose. It allows the filling of the vacuum area rapidly and efficiently without leaving gaps that reduce the efficiency of suction. Large vacuum cavities are difficult to fill and especially in vivo, where the tissue has an asymmetric and irregular profile. The irrigation channel 2058 hydrates the tissue and increases the numerical aperture of the imaging objective lens. Water or biocompatible oils can be considered as immersion mediums that are constantly renewed in the imaging area. This allows compensation for the amount of immersion medium that has been removed by the vacuum system, absorption by the tissue, evaporation, etc. In addition to the immersion medium, pharmacologic agents can also be delivered to the mucosal surface.

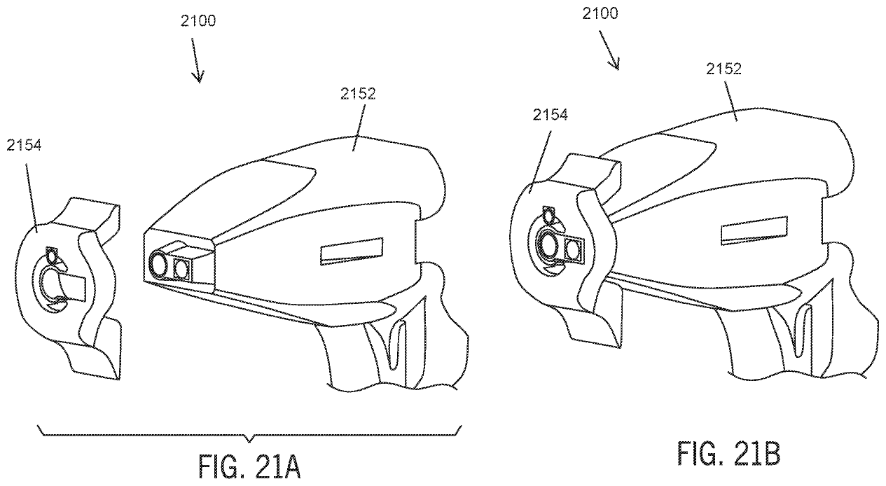

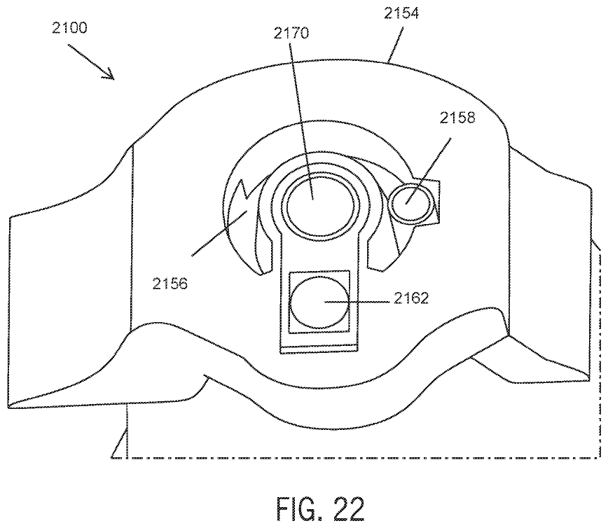

Looking now at FIGS. 21A, 21B, 21C, and 22, there is shown another example embodiment of an imaging instrument 2100 according to the invention with a disposable tip. The imaging instrument 2100 includes a housing 2152 and an imaging section with a disposable tip 2154. The tip 2154 has an annular vacuum channel 2156, an irrigation channel 2158, a light-outputting end 2162 of an optical fiber transmitting light from a light source, and an objective lens 2170. The disposable tip 2154 ensures the portion of the instrument that comes into contact with patients is sterile. The complex design can be enabled by 3D printing.

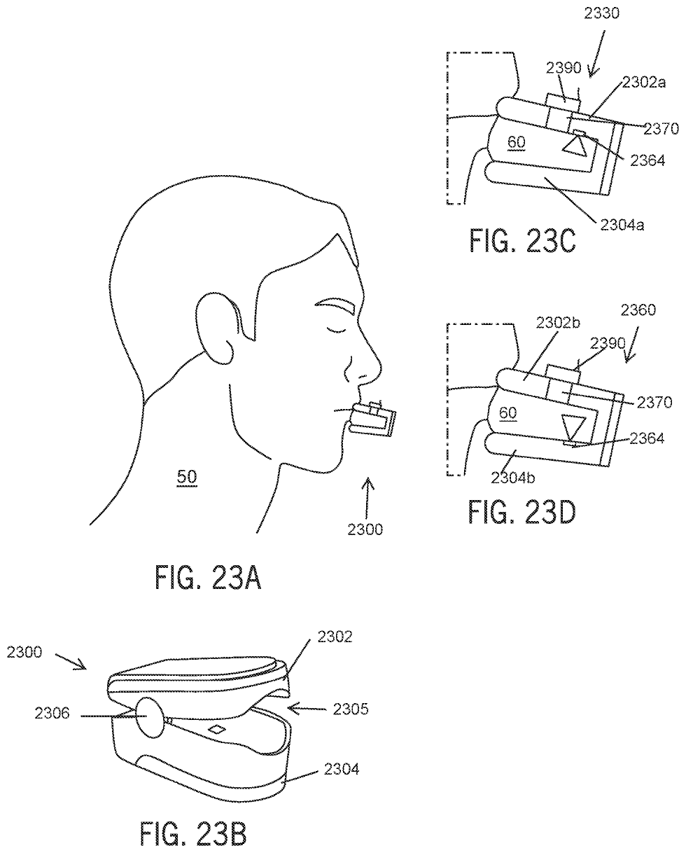

Turning to FIGS. 23A and 23B, there is shown another example embodiment of a portable system 2300 for imaging of microvasculature of tissue according to the invention positioned on a human subject. The portable system 2300 includes a moveable first arm 2302 and an opposed second arm 2304 defining a space 2305. A hinge 2306 connects the first arm 2302 and the second arm 2304.

FIG. 23C shows one version of a portable system 2330 similar to portable system 2300. The portable system 2330 includes a moveable first arm 2302a and an opposed second arm 2304a defining a space that receives the oral mucosa 60. A light source 2364, an objective lens 2070, and an image detector (e.g., sensor/camera) 2390 are located in the first arm 2302a such that the portable system 2330 is in an oblique back-illumination configuration as shown in FIG. 23C.

Looking now at FIG. 23D, there is shown another version of a portable system 2360 similar to portable system 2300. The portable system 2360 includes a moveable first arm 2302b and an opposed second arm 2304b defining a space that receives the oral mucosa 60. A light source 2364 is located in the second arm 2304b, and an objective lens 2070 and an image detector (e.g., sensor/camera) 2390 are located in the first arm 2302b such that the portable system 2360 in an oblique trans-illumination configuration as shown in FIG. 23D.

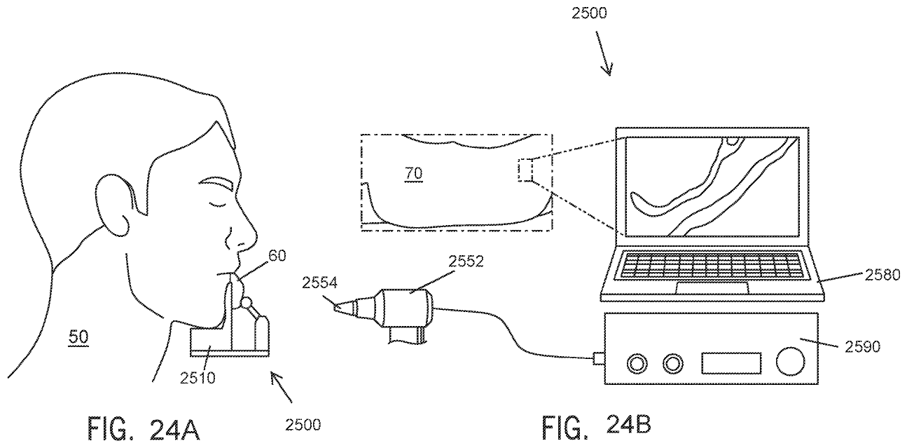

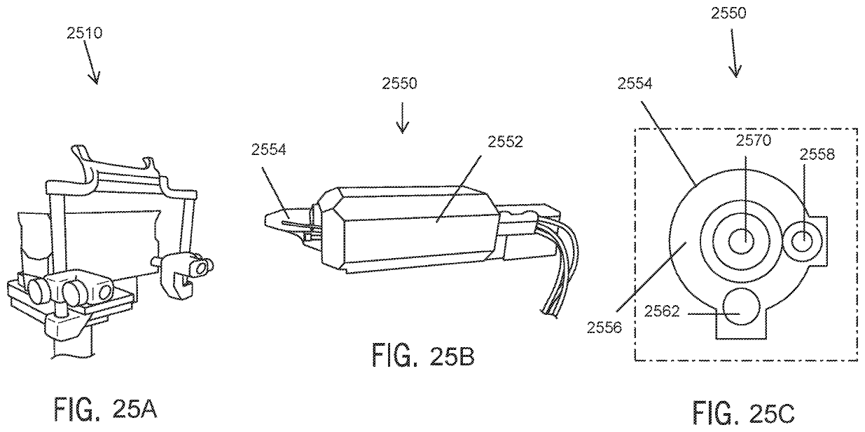

Looking now at FIGS. 24A, 24B, 25A, 25B and 25C, there is shown another example embodiment of a system 2500 for imaging of microvasculature of tissue according to the invention positioned on a human subject 50. The system includes a tissue stabilizer 2510 (similar to tissue stabilizer 500 described above) and an imaging instrument 2550. The imaging instrument 2550 includes a housing 2552 having an imaging section with a tip 2554. The tip 2554 has an annular vacuum channel 2556, an irrigation channel 2558, a light-outputting end 2562 of an optical fiber transmitting light from a light source, and an objective lens 2570. The imaging instrument 2550 includes a controller 2580 which may include a memory and an electronic processor coupled to the memory.

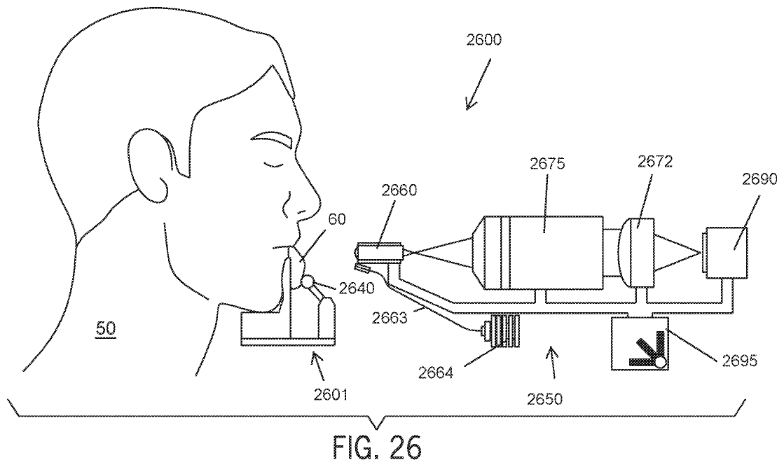

Turning to FIG. 26, there is shown another example embodiment of a system 2600 for imaging of microvasculature of tissue according to the invention positioned on a human subject 50. The system 2600 uses a microscope objective. The system includes a tissue stabilizer 2601 with an adapter 2640 (similar to tissue stabilizer 500 described above), and an imaging instrument 2650. The imaging instrument 2650 includes a LED light source 2664, a light transmitting optical fiber 2663 having a light-outputting end, a GRIN objective lens 2660, a microscope objective 2675, a double achromat lens 2672, a CMOS camera 2690, and a motorized XYZ system 2695 for movement of the imaging instrument 2650.

The tissue stabilizers can comprise materials that are biocompatible, sterile or sterilizable. A single stabilizer can comprise multiple materials (e.g., plastic+glass, metal+plastic, polymerized resin+metal+glass, etc.). The material can be biocompatible: metal (stainless steel, titanium, etc.), plastic, polymer, ceramic biomaterial, 3D printing material (resin or photo-resin), silicone, short-term and long-term implantable, etc. The material can be: sterile: material(s) sterilized by the manufacturer; single-use per patient; disposable; sterilizable: material(s) that can be sterilized by the user (e.g., certified researcher, clinician, etc.); and multiple use and sterilizable by chemical sterilant, radiation, wet heat, dry heat, etc.

The systems of the present invention have many advantages. The miniaturized imaging instruments (see FIGS. 15-26) enable noninvasive, label-free visualization and recording of the microvascular content of the oral mucosa tissue in vivo. To generate contrast between various types of biological tissues and cells, the instruments of the invention combine:
1. Phase-gradient contrast. This can be obtained by oblique illumination of the tissue, and allows visualization of fine morphological details of the tissue (e.g., membrane) and blood cells (e.g., membrane, granularity, nucleolus, etc.) that are inaccessible with existing commercial devices (CytoCam, MicroScan, etc.).
2. Absorption contrast. This is enabled by using a specific wavelength range of light (530+/−20 nm.) that is strongly absorbed by red blood cells (which carry hemoglobin) compared to white blood cells. This also has the potential to help differentiate oxygenated vs. non-oxygenated red-blood cells.

The systems of the present invention have many purposes including, without limitation:
1. real-time imaging and characterization of the morphology of the oral mucosa tissue;
2. real-time imaging and characterization of the oral mucosa's microvasculature and its content;
3. quantification of blood content (e.g., white and red blood cells, platelets, etc.);
4. characterization of blood vessel and blood cell morphology/shape; and
5. characterization of blood cell movement.

The oral mucosa stabilizers are non-exclusive to the miniaturized instruments, and can also be considered in benchtop imaging configurations: multi-photon systems, pulsed laser systems, scanning systems, etc.

### Methods of Software Operation

Systems of the invention can include image data processing. Software is capable of identifying, counting, and tracking the microvascular, cellular content from recordings acquired from the imaging system. Its operation can be fully automated, semi-automated or manual. Its code may be written in any existing programming language (e.g., Python, C++) as executable scripts or programs, and/or as scripts and macros for any existing commercial or open-source software including but not limited to: ImageJ, Fiji, MATLAB, Imaris, etc. Purposes of the software include: (1) reporting the cell count per volume for different blood cell types in the microvasculature, and the movement type of the cells, and (2) reporting the perfusion characteristics of the imaged vessel, as a quality measure of (1). See Example 2 below for a pipeline employing methods #2-7.

### Image Analysis Method #1: Intensity Profile

This method is based on the extraction of the temporal intensity profile within the selected regions of interest (ROI) in the field of view. The temporal intensity recording from a single or multiple ROIs can be used for the following purposes:
extract blood flow and perfusion parameters;
measure heart rate;
detect the presence of white blood cell (WBC) candidates in the circulation or on the blood vessel walls;
verify the detection of WBC candidates (by using multiple ROIs at different locations on the vessel); and
reduce the background of the image.

### Image Analysis Method #2: Region-of-Interest Selection

This method designates a volume within a video for microvascular content analysis. The designated volume is free of non-vascular pixels.

Attributes of the pixels that may be used as the basis of designation include, but are not limited to: intensity, change in intensity a function of time, intensity gradients along the edges.

Any algorithm that measures and segments a given image by these attributes may be used. User input may be requested to verify and correct the output of automated algorithms.

The output of region-of-interest selection includes, but is not limited to: a designated volume for which microvascular content analyses can be performed

### Image Analysis Method #3: Coordinate Transformation

This method creates a transformed coordinate system that preserves the geometry of the microvascular regions-of-interest (ROI). The main feature of such a transformed coordinate system is that their two axes run parallel and normal to the blood flow respectively.

The axis parallel to the blood flow, x′, is defined using skeletonization. Skeletonization is a classical image processing algorithm that reduces the foreground of a binary image to a “skeleton” that best captures the foreground's key attributes including curvature, connectivity, and extent. For foregrounds sharing the shape of a blood vessel, the skeleton is a line that follows the axis of the vessel and bends in accordance to the local curvature.

Additional gridlines along x′ are drawn using the skeleton as reference, thereby also capturing the microvascular ROI's morphology. Image processing morphological operations (e.g. dilations and erosions) may be used to perform this step.

The axis normal the blood flow, y′, is defined by the normal lines of the skeleton. Each of these normal lines intersects the skeleton, and has a slope perpendicular to the skeleton's at the intersecting point.

The transformed coordinate system permits analyses of microvascular content as a function of space (x′,y′) and time (t). Units of measurement can be defined along the three axes. The software includes pre-optimized but user-definable values for these distance and time units. Vessel block, a unit along x′, is defined by the number of pixels along the skeleton. y′-gridline index, a unit along y′, refers to the particular y′ gridline along which the measurement is made. Time segment, a unit along t, is specified by the number of video frames.

The output of coordinate transformation includes, but is not limited to:
the transformed coordinate system;
units of microvascular content measurement, defined using the transformed coordinate system; and
morphological parameters of the microvascular ROI (e.g. length, width, cross-section area, volume, curvature).

### Image Analysis Method #4: Digital Line Scans

This method pertains to the use of digital line scans to create space-time diagrams, from which perfusion parameters can be extracted. Space-time diagrams, generally defined as 2D plots with time as one axis and distance along a dimension as the other, show the position of objects as a function of time.

A space-time diagram can be generated for each unit of measurement, i.e. for each vessel block, y′-gridline index and time segment. The y-axis of these space-time diagrams represent time elapsed from the start of the time segment, and the x-axis, the pixel length along the skeletons for the given vessel block.

Leukocytes and other high intensity objects travelling along the skeleton, or any of the y′-grid lines running in parallel to the skeleton, appear as bright, sloped bands in the corresponding space-time diagram. The gradient of these bands corresponds to the velocity of the objects.

The width of the bands in space-time diagrams are related to object size. Digital image filters may be applied to the diagrams to extract the velocity values of objects of a certain size range.

The time of appearance of the object within the unit of measurement can be determined by taking the cumulative sum of pixel intensities along the slope of the bands. Such summation can be performed with sliding windows of different lengths, can be processed in combination with different image processing algorithms to ensure small leukocytes will have an equal chance of detection, and deviations of travel speeds from the blood flow velocity value are tolerated.

The output of digital line scans includes, but is not limited to:
space-time diagram for each unit of measurement;
velocity of bright objects, which include candidate leukocytes, traveling within the unit of measurement; and
intensity profile, as a function of x′ and t, within the unit of measurement.

### Image Analysis Method #5: Consolidation

This method describes the process of assembling the output from multiple units of measurements, each specified by its vessel block, y′-gridline index, and time segment, and groups the output by their source object.

A flowing bright object in the vasculature, e.g. a candidate leukocyte, is expected to be captured by multiple time segments, with a temporal delay along x′, the direction of blood flow that reflects the time needed for the object to travel downstream from one unit of measurement along x′ (vessel block) to the next. The flowing object may or may not span the full width of the vessel, i.e., it may be captured by certain y′-gridlines and not others. Consolidation considers these factors.

Consolidation is performed on all three axes of analysis: (x′,y′) of the transformed coordinate system, and time t. Consolidation along y′, by y′-gridline indices, uses a sorting-by-pixel strategy and ensures the capturing of all flowing objects, regardless of their relative size compared to the full vessel width. Consolidation along t assembles the information from all time segments such that it spans the full length of the video. Consolidation along x′, by vessel blocks, aligns the information from different vessel blocks by matching the flowing objects identified in one vessel block to the flowing objects identified in another vessel block.

Inter-vessel-block matching of flowing objects may deploy any of the image processing methods that perform peak matching, including but not limited to dynamic time warping (DTW) and cross-correlation. The output of inter-vessel-block matching is a collection of matched intensity profiles, one for each vessel block, in which the peaks are matched in position. The peak shifts required for the matching to occur is related to the time delay for each flowing object to travel from one vessel block to another, i.e. its velocity.

An index number is assigned to each set of matched peaks between vessel blocks. Each index number identifies one source object, e.g. a candidate leukocyte, likely to contribute to the set of matched peaks. The number of these indices, called “gate indices”, is the candidate leukocyte count estimate.

The output of consolidation includes, but is not limited to:
consolidated velocity, as a function of x′ (the direction of blood flow) and time;
consolidated intensity profiles, one for each vessel block, as a function of time;
a list of “gate indices”, each referring to a candidate leukocyte; and
the candidate leukocyte count estimate.

### Image Analysis Method #6: Gating

This method specifies the spatial and temporal volumes in which flowing objects, e.g. candidate leukocytes, appear in the video. One gate corresponds to one candidate leukocyte, which is assigned an index number, a “gate index”, during consolidation.

Gating prepares for the trimming of the video into a collection of shorter-in-duration and smaller-in-area videos for the confirmation of leukocyte presence and final leukocyte counting. Each of these shorter, smaller videos approximates the volume of appearance of a candidate leukocyte. Since leukocyte concentration is usually low in blood (˜0.15% of red blood cell concentration in healthy individuals), this trimming can greatly expedite the leukocyte confirmation and final counting process. As a method, gating takes the candidate leukocyte information from consolidation, defined in the transformed coordinate system and time, and convert them into the video's (cartesian) coordinate system and time. The output is a volume (“gate”) for each candidate leukocyte, delineated by bounding boxes in a consecutive series of video frames that specify the spatial and temporal location of the cell's appearance in the video.

The output of gating includes, but is not limited to:
Bounding box coordinates corresponding to the volumes of appearance of the candidate leukocytes in the video, organized by gate indices.

### Image Analysis Method #7: Detect-and-Link Particle Tracking

Leukocyte detection and counting may be performed using the detect-and-link method for particle tracking. In this method, particles in individual frames of a video are first detected. Detected particles from consecutive frames are subsequently linked to track their movement. The number of tracks determines the particle count.

This method can be executed on its own, or as part of a pipeline with the volumes of candidate leukocytes already reported, in which case this method serves to confirm the presence of the already identified candidate leukocytes, and verify that their count equals the candidate leukocyte count estimate. While the bright bands in space-time diagrams are mostly leukocytes, they may, in rare cases, be stretches of plasma free of flowing blood cells, especially in a narrow microvascular ROIs. Furthermore, leukocytes may appear as multiplets (doublets, triplets etc.), in which case one gate index represents more than one leukocytes.

The input of this method may therefore be the full video, or videos cropped from the full video using the spatial and temporal coordinates supplied by gating.

### (i) Detect

Particles in this method are blood cells found in the microvasculature. Detection of these particles, or cells, may be performed manually, semi-automatically or automatically using any of the algorithms that identify foreground and background with different intensities.

Such algorithms may be those from classical image processing, including but not limited to those purposed for blob detection (e.g. determinant of Hessian, Laplacian-of-Gaussian), and segmentation (e.g. thresholding, graph cut, WEKA®).

Such algorithms may also be from computer vision methods. The convoluted neural network (CNN) is a deep learning algorithm capable of learning features in images. Different CNN layer architectures, including but not limited to different versions of squeezeNet, ResNet, EfficientNet, MobileNet, R-CNN, and YOLO may be used. Modifications and combinations of this architecture may also be used. The CNN models are first trained to recognize and localize the blood cells. The same or a separate model can also be trained to identify the subtype (e.g. granulocytes, lymphocytes) of each cell.

The training data of CNN models provided in the software are videos of the microvasculature in the oral mucosa tissue, recorded with similar acquisition parameters as the videos to be analyzed. Videos of both healthy and diseased patients are used for training. Weights and biases of the models are therefore optimized for the imaging system and the application. Transfer learning may be used for the training of CNN models.

The output of detection includes, but is not limited to:
the detected cell(s)' locations, which may be reported as a list of pixels or as bounding boxes in each video frame;
the cell(s)' subtype; and
the confidence score of the above predictions.

### (ii) Link

The software provides estimates of motion parameters for each cell motion type (e.g., free-flowing, rolling, crawling, tethering) to ensure proper linkage. These parameters include, but are not limited to, velocity, preferential location inside the vessel lumen, and directionality.

To address the issue of cells being intermittently concealed in the videos by other cells, particle linking algorithms may be performed with or without state estimation. The software also provides optimized parameters for such algorithms, including but not limited to Kalman filters and Particle filters.

The output of linking includes, but is not limited to:
count of cells, with the option of count by subtype (e.g. granulocytes, lymphocytes) in the input video;
motion track of each cell in the input video; and
motion type (e.g., free-flowing, rolling, crawling, tethering) of each cell in the input video.

### Summary of Information Obtained From the Software

The following information may be included in the summary report generated by the software:
blood flow parameters (e.g., flow speed, blood volume, etc.);
number of leukocytes;
subtype of leukocytes (e.g., granulocytes, lymphocytes);
motion type of leukocytes (e.g., free-flowing, rolling, crawling, tethering) and parameters of the motion (e.g., speed, acceleration, etc.);
blood vessel morphology (endothelium, glycocalyx layer, etc.); and
heartbeat.

### EXAMPLES

The following Examples are provided to demonstrate and further illustrate certain embodiments and aspects of the present invention and are not to be construed as limiting the scope of the invention. The statements provided in the Examples are presented without being bound by theory.

### Example 1

### Overview of Example 1

A miniature oblique back-illumination microscope (mOBM) for imaging the microcirculation of human oral mucosa, enabling real-time, label-free phase contrast imaging of leukocyte rolling and adhesion, the initial steps in leukocyte recruitment that is a hallmark of inflammation. Imaging cell motion can provide new diagnostic information (time course of disease progression, response to therapy, etc.) that is not available using traditional static diagnostic parameters such as a cell number and morphology.

The ability of leukocytes to traffic to various organs and tissues is a fundamental requirement for proper immune function [Ref. 1]. Leukocyte recruitment is a multi-step process initiated by the slowdown of circulating leukocytes that tether and roll on the endothelial surface, followed by their firm adhesion and extravasation into tissue [Ref. 2]. Though well characterized in animal models using intravital microscopy, the rolling and adhesion events (collectively known as leukocyte-endothelial interaction, or LEI [Ref. 3,4]) have rarely been observed in humans [Ref. 5]. Conceptually, imaging cell motion as a potential source of diagnostic information has yet to be explored in clinics, as traditional histopathology has relied on the static examination of biopsied samples. LEI is reported to be significantly increased in the sublingual microvasculature of patients with systemic inflammation such as sepsis [Ref. 6] and ischemia-reperfusion injury [Ref. 7]. However, assessing LEI in clinical settings has been challenging due to the lack of proper detection and analytical tools. Individual leukocytes are not resolved using existing clinical instruments such as CytoCam and MicroScan [Ref. 8]; instead, their presence is inferred from the gaps or voids in the blood vessels otherwise filled with red blood cells. The suboptimal image quality is further compounded by the severe motion and pressure artifacts and a lack of proper analytical tools to quantify leukocyte motion [Ref. 9]). Reflectance confocal microscopy (RCM) provides high-resolution cellular imaging and has been successfully used in dermatology clinics [Ref. 10]. However, RCM requires laser scanning with limited frame rate [Ref. 11]. Nonlinear optical techniques such as third-harmonic generation (THG) microscopy [Ref. 12] and two-photon-induced UV autofluorescence imaging [Ref. 13] can also provide label-free imaging of leukocytes but these techniques entail complex laser systems and scanning platforms with no clear path for translation to the bedside.

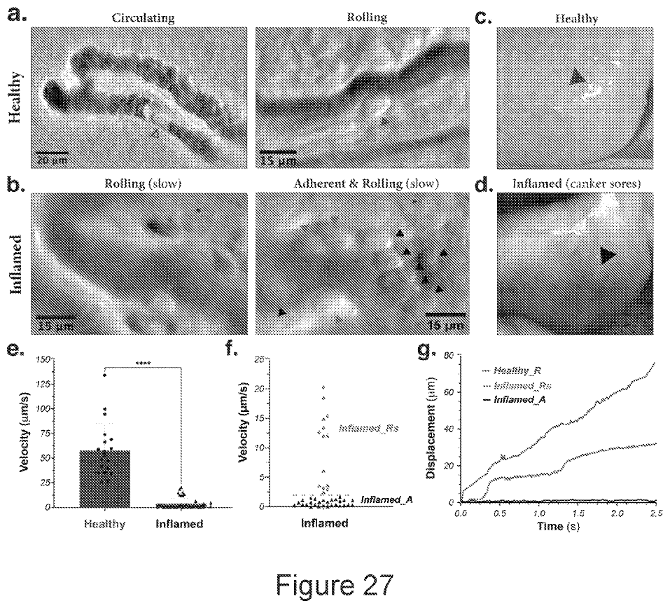

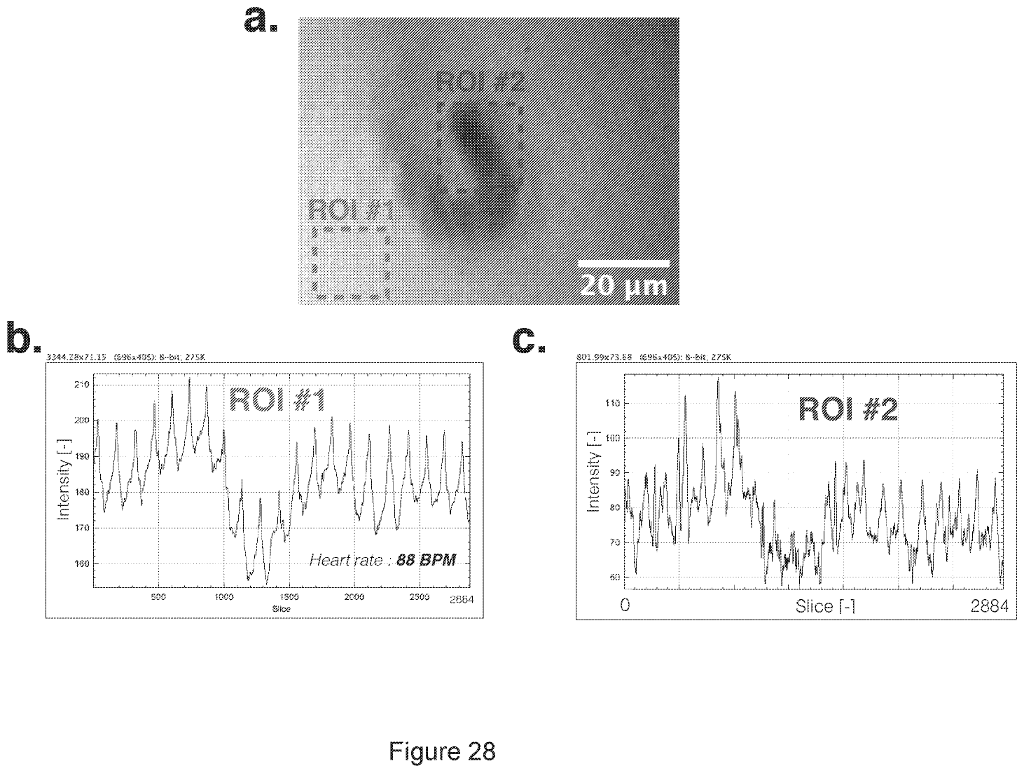

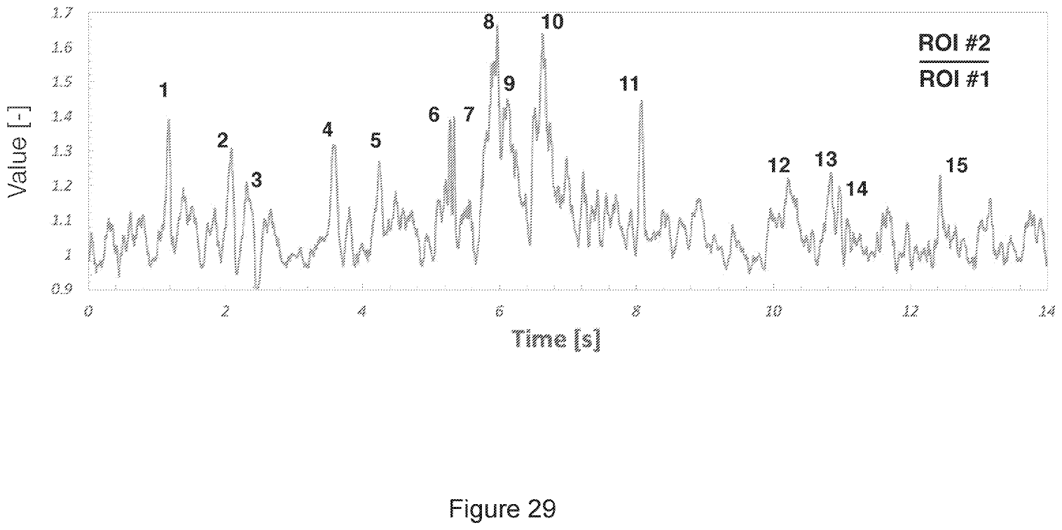

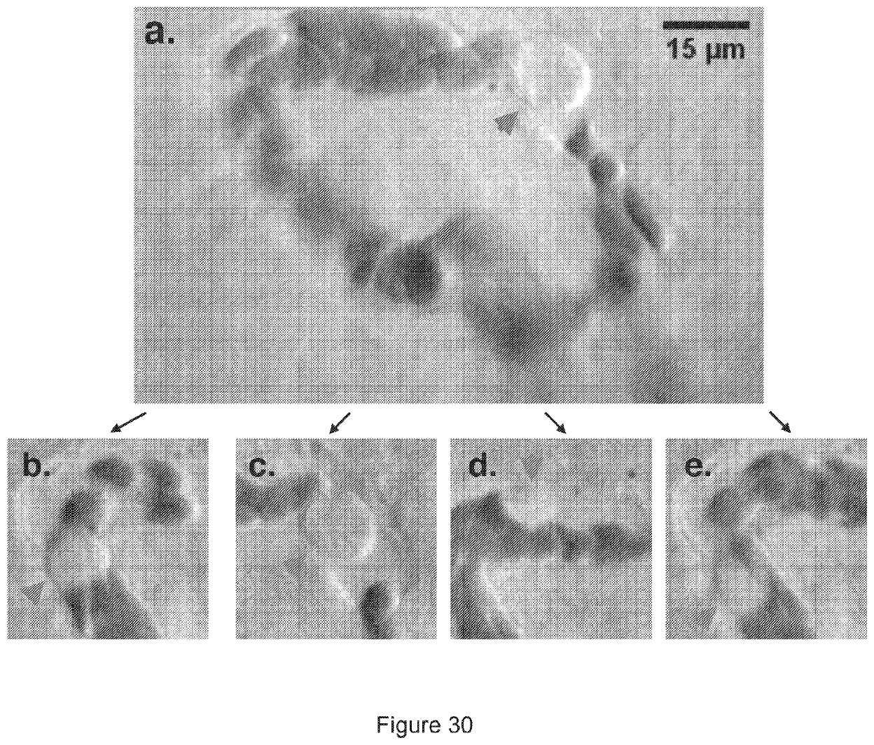

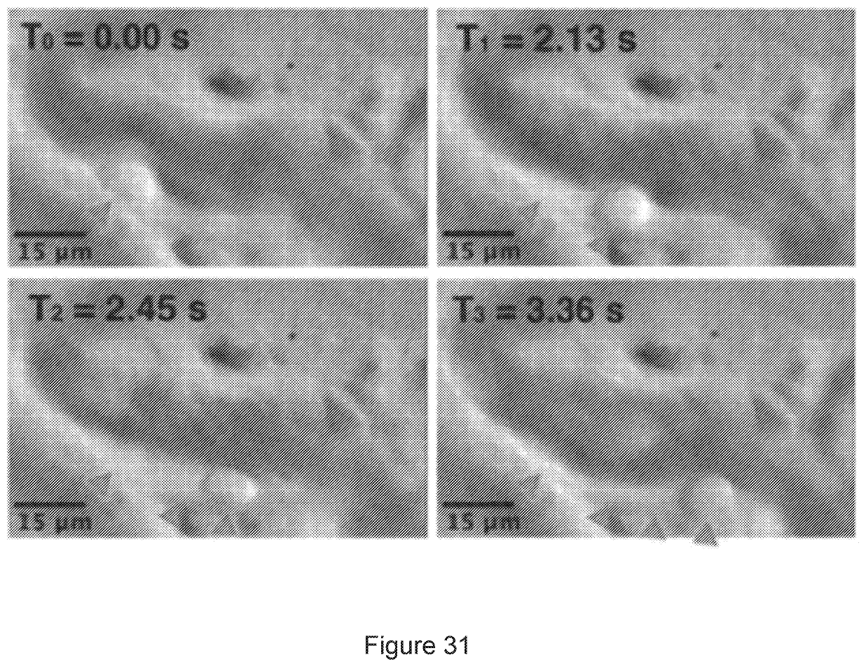

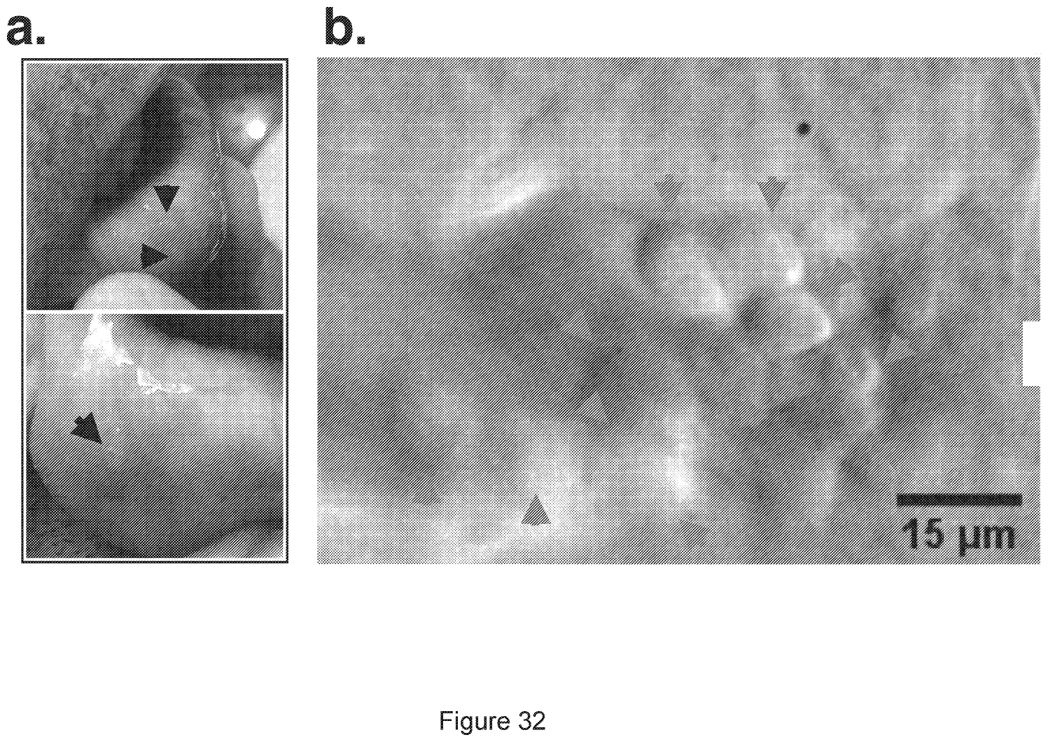

To address these limitations, we developed a miniaturized oblique-back illumination microscope (mOBM) for non-invasive imaging of the microvasculature of human oral mucosa (FIGS. 24A, 24B, 25A, 25B and 25C). The mOBM (FIG. 25B) comprises a 1 mm diameter aberration-corrected gradient index (GRIN) objective lens with a numerical aperture (NA) of 0.75 and a green LED (light emitting diode) light source coupled to a large core (Ø 1 mm) multimode optical fiber. The output end of the fiber is positioned such that photons enter the tissue from one side of the GRIN lens (FIG. 25C). Injected photons undergo multiple scattering events in deep tissue layers, and only a fraction of the photons are collected by the GRIN lens, providing the back-illumination at an oblique angle due to the offset geometry of the optical fiber [Ref. 14]. The asymmetric (oblique) back illumination generates phase-gradient contrast (PGC) images that resemble differential interference contrast (DIC) [Ref. 15] or differential phase contrast (DPC) [Ref. 16] microscopy images with a crucial difference. Unlike DIC or DPC, OBM is designed to work with intact, thick tissue [Ref. 14] and is therefore compatible with in vivo imaging. The use of the green LED additionally provides contrast based on hemoglobin absorption, making the white blood cells stand out against the darker red blood cells (FIG. 27 Panel a, left).

To minimize motion and pressure artifacts, a custom tissue stabilizer (FIG. 25A) is developed to gently expose the lower lip's inner microvasculature (FIG. 24B) and to maintain its position for the duration of the imaging. The choice of the inner lip is based on its ready access, its well-developed shallow vascular bed, its lack of skin pigmentation, the absence of the highly scattering stratum corneum layer that degrades the image quality in the skin, and the comfort of participants. To validate the performance of our system we imaged the movements of leukocytes in the mucosal microvasculature of human volunteers. When imaged at a frame rate of 200 Hz, individual blood cells in the microcirculation are clearly delineated (FIG. 27 Panel a, left). These cells travel at a speed of ˜1 mm/sec, and their motions are effectively “frozen” when the exposure time of individual frames is set to <1 msec. In addition, rolling cells are detected in a subset of blood vessels of healthy volunteers (FIG. 27 Panel a, right), likely postcapillary venules based on their diameter and flow speed.

We also imaged an inflamed area caused by the presence of canker sores (FIG. 27 Panels b-d), and observed a drastic reduction in the average speed of leukocytes. Using automated frame-by-frame leukocyte tracking (see Methods below), we obtained an average rolling velocity of 58±28 μm/s in the healthy tissue (FIG. 27 Panel e), which reduced to 4±6 μm/s in the inflamed tissue (FIG. 27 Panel e). Closer inspection revealed two populations (FIG. 27 Panel f), an adherent population with an average velocity about 0.7±0.7 μm and a slowly rolling population with an average velocity of 11±6 μm. The adherent cells were restricted to the area close to the canker sores (FIG. 27 Panel d, black arrowheads), while the slowly moving leukocytes were detected both in the center and in the periphery (FIG. 27 Panel d, red arrowheads) of the inflamed area. The displacement profile showed the characteristic stop-and-go movement patterns during inflammation (FIG. 27 Panel g).

To make the instrument even more compact, the current focusing mechanism (a translational stage) can be replaced with an electrically tunable lens [Ref. 17]. In addition, the high-speed CMOS sensor (frame rate up to 1000 Hz) can be replaced by a standard (30 Hz) video camera that will still be able to image rolling and adherent cells, but the flowing cells will not be resolved at this frame rate. The high-speed imaging capability offers the tantalizing possibility of performing noninvasive white blood cell count by resolving individual circulating cells and flagging leukocytes “on the fly” with the help of machine learning, a subject of active pursuit in our laboratory and others [Ref. 18]. Because of its compact size, low cost, and simple construction, we expect to place the mOBM instrument in several clinics to begin testing the diagnostic utility in critically ill patients and preterm infants at high risk of infection and sepsis. We further envision the instrument to find use in resource-poor settings that lack the expertise and infrastructure to draw blood for standard laboratory analysis.

### Methods

Features of the Developed System. The optical design of mOBM was accomplished using the Zemax software (FIG. 18A). It comprises a 1 mm diameter gradient index (GRIN) objective lens, relay optics, and a CMOS camera. The imaging GRIN lens assembly (Grintech, GT-MO-080-032-ACR-VISNIR-08-20) is aberration-corrected and has a high numerical aperture (NA) of ≈0.75 (in water). The relay optics comprises a GRIN lens (Edmund Optics, #64-519) with a ≈¼ pitch and NA=0.52 together with an achromat doublet lens (Edmund Optics, #49-772) with an effective focal length of 19.1 mm that projects the magnified image onto the CMOS sensor (Basler, daA1920-160 um). Oblique illumination is provided by a 530 nm LED light source (Thorlabs, M530F2) coupled to a 1 mm diameter large core optical fiber, whose output (≈19 mW) end is positioned on one side of the GRIN objective lens assembly (FIG. 25C). The developed mOBM (FIG. 25B) detects multiply scattered photons, in a non-confocal manner, generating phase-gradient contrast (PGC) images that reveal fine morphological details (e.g., cell membrane, granules). In addition to the PGC, our instrument also benefits from absorption-contrast generated by using the 530 nm wavelength that is strongly absorbed by hemoglobin (in red blood cells) and not by leukocytes. An important characteristic is the imaging tip (FIG. 25C), which comprised four channels: 1. A central channel containing the miniaturized GRIN objective lens. 2. An illumination channel containing the large core multi-mode fiber. 3. An irrigation channel to keep oral mucosa tissue moist and to maintain the immersion medium in front of the objective lens. 4. A ring-shaped vacuum cavity surrounds the central channel to stabilize the imaging area. The magnification of our instrument was ≈25, which resulted in ≈7.2 pixels/μm (without binning) and ≈3.6 pixels/μm (with 2×2 binning) ratios. The field-of-view of our system was ≈150×200 um and the working distance of the objective lens was adjusted (20-100 μm) by means of motorized displacement of the camera in the direction of the optical axis. SolidWorks software was used to design mOBM's mechanical housing, the imaging tip, and the oral mucosa apparatus (FIG. 25A). Parts were printed using a 3D laser printer (Formlabs 3B). All parts in contact with the human tissue were printed with sterilizable, biocompatible materials (Formlabs, RS-F2-BMCL-01, RS-F2-BMBL-01).

Tissue Stabilizer. The developed universal oral mucosa apparatus (FIG. 25A) was used to gently hold the subject's lower lip tissue and expose buccal microvasculature (FIG. 24B) without generating undesired blood flow alternations. The apparatus also stabilized the tissue for the duration of the imaging session (≈20-60 minutes). A motorized XYZ stage was used to position the imaging tip of the mOBM and locate the microvasculature of interest.

Imaging of Healthy Participant. Subjects were first seated in front of the imaging system and their head was gently stabilized using soft tissue straps. The lower lip was then enrolled using the developed oral mucosa apparatus (FIG. 25A). There were no additional requirements from the subjects other than remaining in a seating position for the duration of the imaging. The imaging session was initiated by the operator who positioned the imaging tip over the region of interest (ROI) using a motorized XYZ actuator with micrometer resolution. The imaging session usually lasts between 20-60 minutes.

Image acquisition parameters. For the circulating leukocytes (FIG. 27 Panel a, left), the acquisition frame rate was fixed at 200 fps and the exposure time varied between 0.5-1 ms. If necessary, 2×2 binning was used to increase the signal-noise ratio. For the rolling leukocytes (FIG. 27 Panel a (middle) and FIG. 27 Panel b), the acquisition frame rate was fixed at 200 fps and the exposure time varied between 2.5-4 ms.

Image Processing Pipeline. Image processing was performed in ImageJ (open-source software). The image processing pipeline for the rolling and adherent leukocytes: 1. Registration (plug-in: Template Matching). 2. Cropping the region of interest (ROI). 3. Blood flow smoothing (plug-in: Kalman filter). 4. Extraction of PGC (Image—Gaussian blurred image (sigma radius: 20-40)). 5. Average subtraction (PGC stack—averaged image) 6. Leukocyte tracking (plug-in: TrackMate). Leukocytes that detached from the endothelial wall after brief contact(s) were excluded from the analysis. To make consistent LEls measurements, we have analyzed rolling cell movement in straight and low curvature vessels with laminar blood flow profiles. Blood vessels with large curvatures and bifurcations susceptible to having turbulent (and therefore unpredictable) blood flow profiles were excluded from the analysis. The image processing pipeline for the circulating leukocytes: 1. Registration (plug-in: Template Matching). 2. Cropping the ROI. 3. Extraction of PGC (Image—Gaussian blurred image (sigma radius: 20-40)).

Statistical analysis. For the statistical analysis (FIG. 27 Panel e) we have used the unpaired t-test method (also known as the student t-test). In the rolling cell group (FIG. 27 Panel e, blue column), n=18; Inflamed group (FIG. 27 Panel e, red column), n=39. P-value <0.0001.

### Example 2

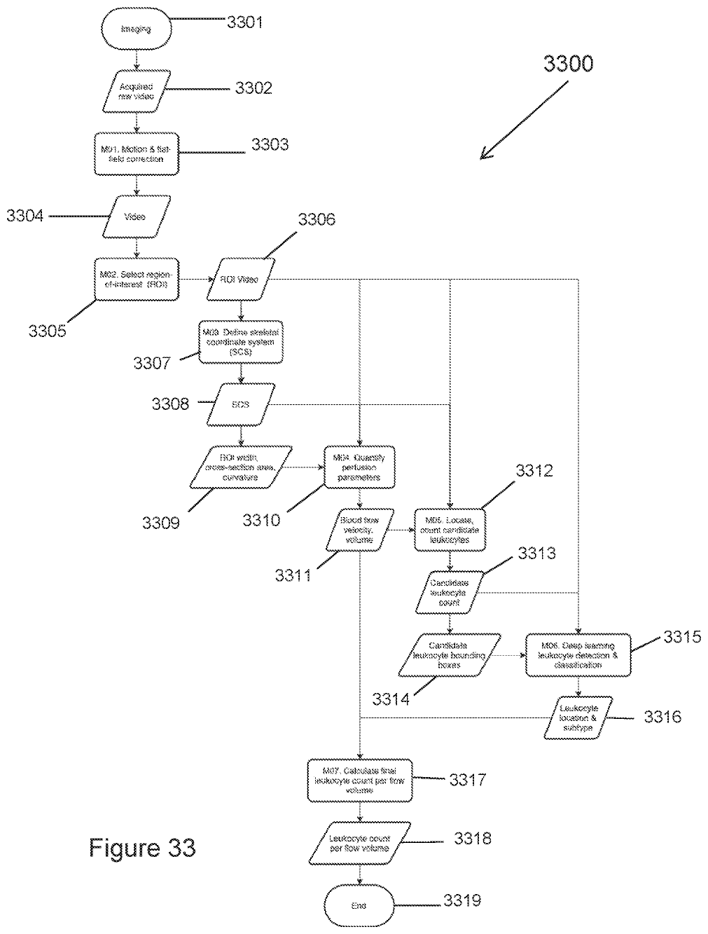

Referring to FIG. 33, an example of a process 3300 for measuring and reporting perfusion parameters and leukocyte counts in videos of the microvasculature acquired by a noninvasive imaging system 3301, which may be one of the systems described herein. In some embodiments, the process 3300 can be implemented as computer-readable instructions on at least one memory and executed by at least one processor coupled to at least one memory.

The process 3300 has seven modules, listed below (M01-M07):
M01. Remove motion artifacts and perform flat-field correction.
M02. Select an appropriate region-of-interest (ROI) for microvascular content characterization.
M03. Define the ROI's Transformed Coordinate System.
M04. Quantify perfusion parameters in the ROI
M05. Locate and provide count estimate of candidate leukocytes.
M06. Deep learning leukocyte detection and subtype classification.
M07. Establish final leukocyte count per flow volume and report findings.

In process step 3301 that begins the process 3300, the video of the microvasculature is acquired by a noninvasive imaging system such as that described above. In process step 3302, the output of 3301, is a video acquired of one or more blood vessels, with leukocytes exhibiting a difference in intensity compared to red blood cells.

### M01. Remove Motion Artifacts and Perform Flat-Field Correction

In the module 3303, image registration is performed to remove motion artifacts in the video introduced by patient movement (e.g. breathing). Uneven illumination is also corrected by background subtraction in the video. In process step 3304, the output of the module 3303, is a video free of motion artifacts and is uniformly illuminated.

### M02. Select an Appropriate Region-of-Interest (ROI) for Microvascular Content Characterization

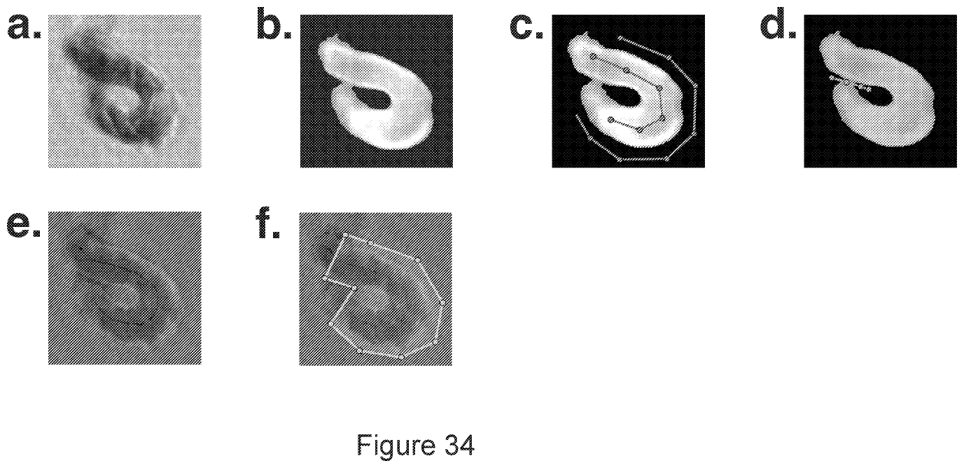

The module 3305 designates a volume for microvascular content analysis. Vessel boundaries are first estimated using a standard deviation time-projection approach (see FIG. 34 Panel b), which effectively separates the pixels that are constant (tissue) or changing (vessel) in intensity over time. The user is asked to confirm the separation (see FIG. 34 Panel c), make corrections to the resulting vessel boundaries as needed (for example, to indicate over- or under-passes; see FIG. 34 Panel d), and finally, to draw a polygon (see FIG. 34 Panel f). The ROI is designated by the pixels that are both within the vascular area (orange) and within the user-drawn polygon (teal). In process step 3306, the output of the module 3305, the video has been cropped to the bounding volume of the ROI.

### M03. Define the ROI's Transformed Coordinate System

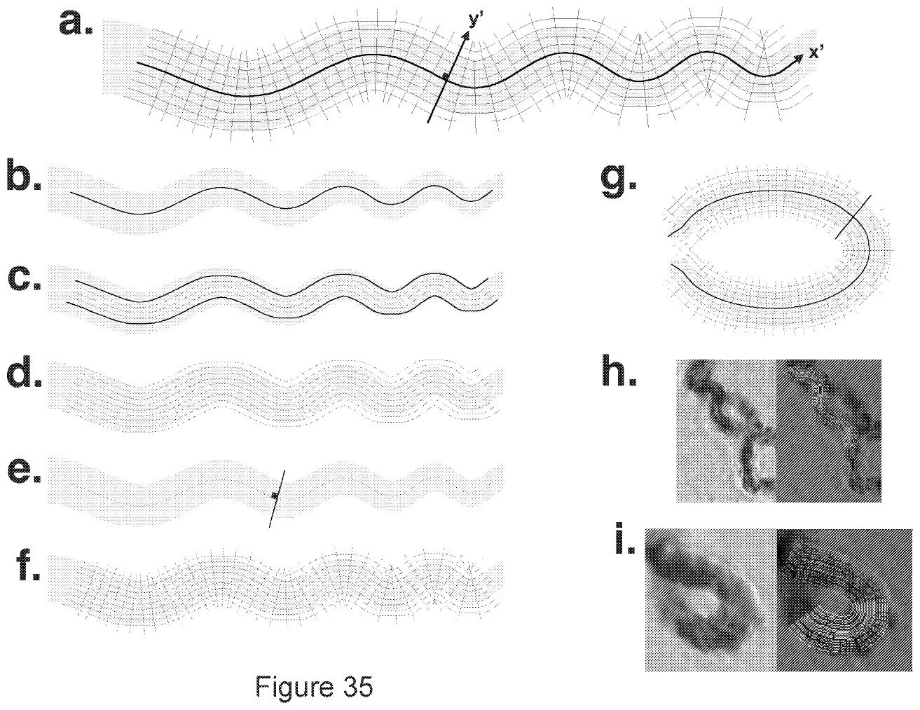

The module 3307 defines a transformed coordinate system for the ROI. The Skeletal Coordinate System (SCS) is one example of a transformed coordinate system. Its two axes, x′ and y′, run parallel and normal to the direction of blood flow respectively (see FIG. 35 Panel a).

The x′ axis is defined using skeletonization. The resulting skeleton co-localizes with the axis of the vessel and mimics its curvature (see FIG. 35 Panel b). The y′ gridlines, which run parallel to x′ axis, are created by duplicating this skeleton to cover the full width of the vessel. Duplication is performed by image dilation and edge detection using structural elements of incrementally increasing sizes (see FIG. 35 Panels c-d).

The y′-axis and x′ gridlines of the SCS are defined by the normal lines of each pixel of the skeleton. These normal lines traverse the skeleton and all its duplicates (see FIG. 35 Panel e-f).

As a transformed coordinate system, the SCS is versatile towards different microvascular ROI morphologies (see FIG. 35 Panel g-i).

In process step 3308, the output of the module 3307, is the transformed coordinate system. In process step 3309, another output of the module 3307, is the collection of morphological parameters of the vessel calculated by the module, including vessel width, cross-section area and curvature.

### M04. Quantify Perfusion Parameters in the Microvascular ROI

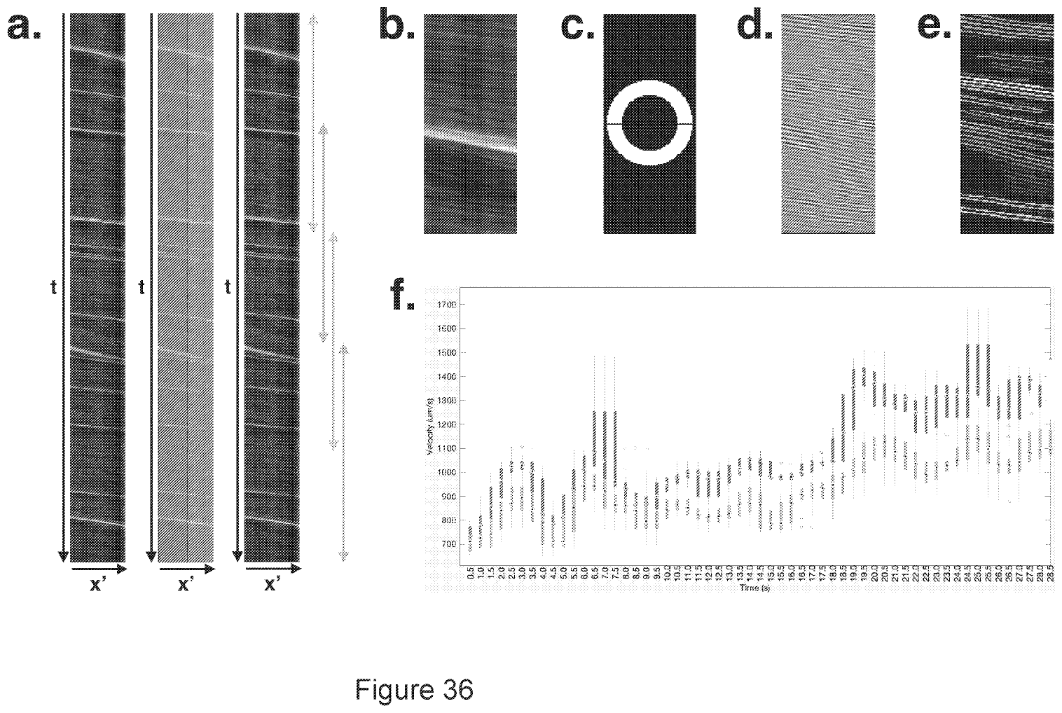

In the module 3310, perfusion parameters of the microvascular ROI are measured for each unit of measurement along x′, y′ and t axis. These units are the vessel block, the y′-gridline index, and the time segment respectively. Each unit of measurement has one corresponding space-time diagram (see FIG. 36 Panels a-b).

Velocity measurements uses only the y′-gridlines near the ROI's axis. Space-time diagrams are filtered in the Fourier space to suppress the bands of noise and larger leukocytes, the latter possibility slowing blood flow (see FIG. 36 Panel c). The space-time diagrams are then converted back to image space and segmented (see FIG. 36 Panels d-e). The angle of each slanted line is measured after the removal of those of insufficient lengths. These angle values are averaged, weighted by their aspect ratio, and converted into one flow velocity value for the space-time diagram.

Consolidation of velocity values from multiple space-time diagrams is performed according to the following rules. If the measurement involves more than one y′ grid lines at any given time point, their mean velocity value is used. If time segments overlap, the mean velocity value of the overlapping time segments is used for each reported time point. The outcome is a plot of flow velocity as a function of time for each vessel block (see FIG. 36 Panel f).

Blood flow volume rate (in volume/time) is calculated by multiplying blood flow velocity and the ROI's cross-section area 3309. Blood flow volume is calculated by multiplying the blood flow volume rate by the duration of the video.

The process step 3311, the output of the module 3310, includes the plot of velocity as a function of time for each vessel block, and the blood flow volume.

### M05. Locate and Provide Count Estimate of Candidate Leukocytes

The module 3312 identifies candidate leukocytes and their approximate temporal and spatial coordinates of appearance in the video.

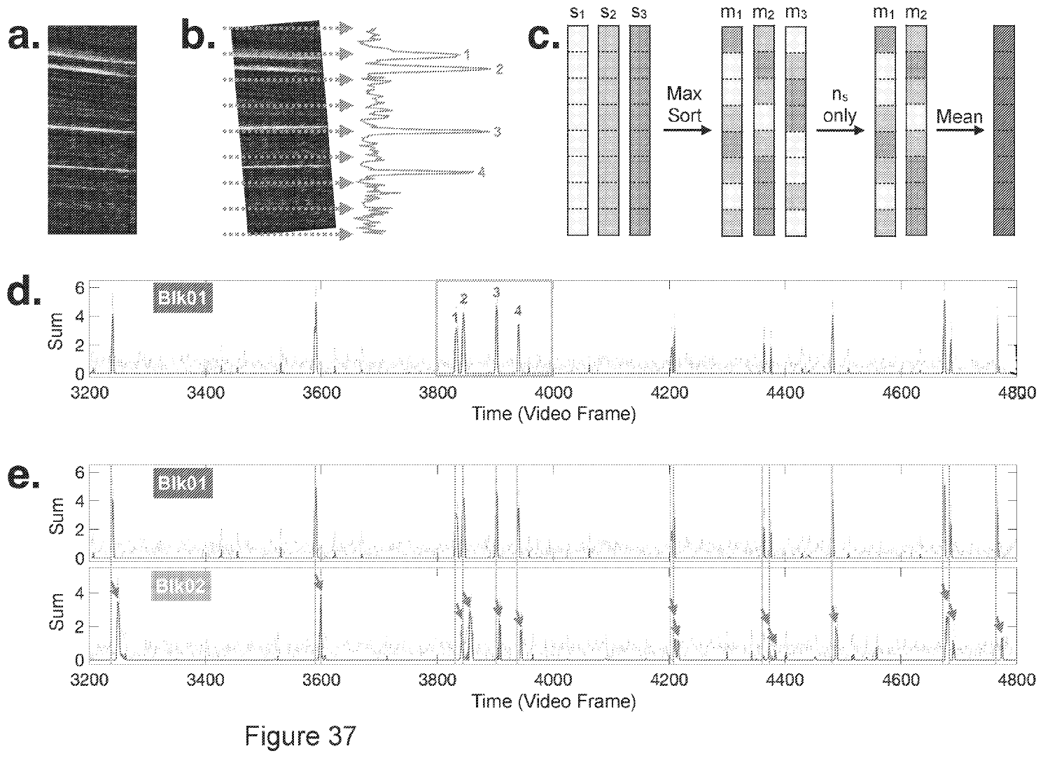

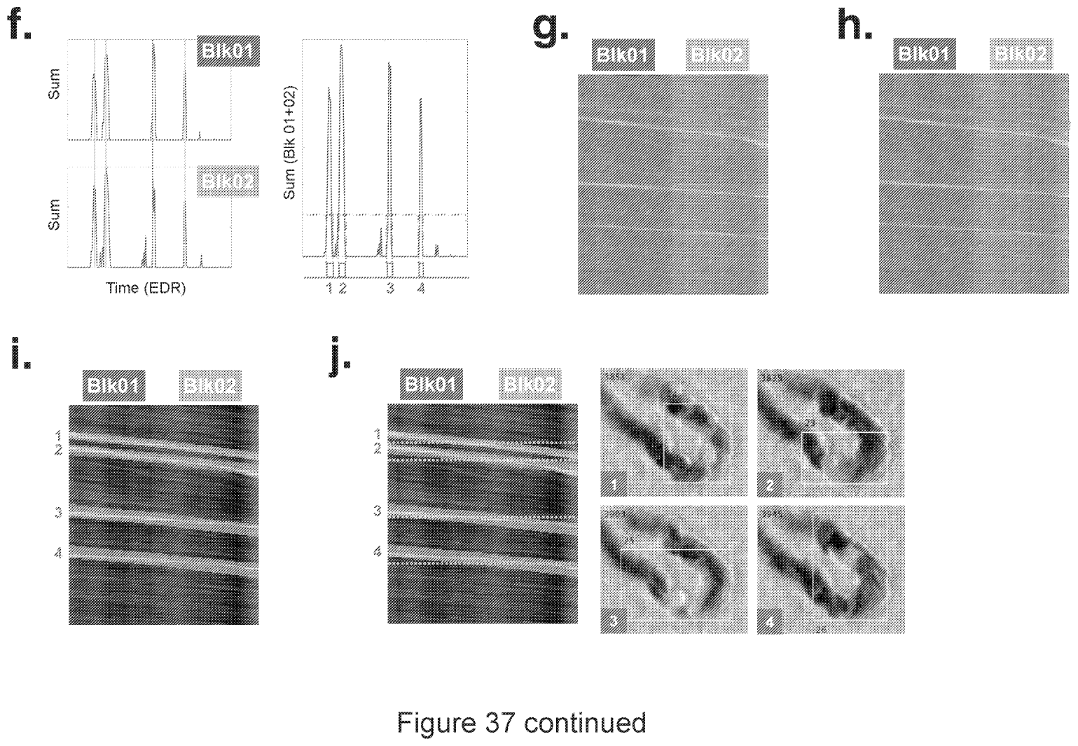

Space-time diagrams are used to locate candidate leukocytes (see FIG. 37 Panel a). Summing along the bands of these diagrams creates a one-dimensional vector in which the high intensity peaks suggest the presence of these cells. A series of moving sums is performed in this example, with a sliding window width ranging from the smallest possible leukocyte diameter to the full width of the space-time diagram. The largest outcome among these sums is chosen as the values of the output intensity vector (see FIG. 37 Panel b).

Consolidation of the intensity vectors gathered from all space-time diagrams is subsequently performed as follows:
i. Consolidation along y′ (by y′-gridlines). Intensity vectors from different y′-gridlines are first combined using a sorting-by-pixel method. For each pixel along the y′-gridlines, the values from all y′-gridlines are sorted in descending order. The result is a set of vectors, in which the first vector contains the maximum by-pixel values of all y′-gridlines, the second vector contains the second largest by-pixel values of all y′-gridlines, etc. High intensity peaks that appear in some but not all y′-gridlines, which indicate the potential presence of small leukocytes, are expected to be captured in the first nₛ vectors, where nₛ is the number of y′-gridlines spanning the smallest possible leukocyte diameter. These nₛ vectors are averaged by pixel to create the new y′-consolidated intensity vector (see FIG. 37 Panel c).
ii. Consolidation along t (by time segments). The y′-consolidated vectors are connected such that the output y′t-consolidated vector covers the full temporal length of the video. Each vessel block has one such output (see FIG. 37 Panel d).

A time delay exists between the y′t-consolidated vectors of consecutive vessel blocks, indicative of the time required for a cell to travel downstream. The time delay is not consistent from peak to peak (see FIG. 37 Panel e).
iii. Consolidation along x′ (by vessel blocks). Peak matching locates the peak in each y′t-consolidated vector that likely belong to the same cell. Dynamic time warping (DTW) is a peak matching algorithm that aligns time series with a similar set of peaks but does not require the individual peaks within the set to share the same offset value. After the peaks are matched in position, the y′t-consolidated intensity vectors of different vessel blocks are summed, completing the consolidation process by creating a y′tx′-consolidated intensity vector that contains peak information from all y′-gridlines, all time segments, and all vessel blocks (see FIG. 37 Panel f).

Gating is performed on the y′tx'-consolidated intensity vector. A threshold is applied, and the peaks exceeding the threshold are characterized for height, and also for their position and width, i.e., their time of appearance in the video. The position and width values of each peak, along with its positional shift between vessel blocks, are used to define the peak's “gate” (see FIG. 37 Panels f-h), defined as the approximate spatial and temporal volume in which a candidate leukocyte appear.

The volumes of all peaks, i.e. of all candidate leukocytes, are converted from the transformed coordinate system to the cartesian coordinate system of the video (see FIG. 37 Panel j). The process step 3314, the output of the module 3312, are these volumes, each identified by a “gate index” that corresponds to a candidate leukocyte. The process step 3313, another output of the module 3312, is the number of such volumes, or “gates”, which is also the candidate leukocyte count estimate.

### M06. Deep Learning Leukocyte Detection and Subtype Classification

The modules M06 3315 and M07 3317 confirm the presence of candidate leukocytes identified in module 3312 by the “detect-and-link” method of particle tracking, and ensures the accuracy of the final leukocyte count. This module 3315 is concerned with “detect”.

The module 3315 first prepares input images appropriate for the deep learning model. The video 3302 is processed by two different background removal methods, and the input and two outputs are merged into a 3-channel video that is cropped according to the gated volumes of candidate leukocyte appearance 3314. Other requirements for the deep learning model, such as tiling of the cropped images, are also performed.

The input images are subsequently fed into a deep learning model pre-trained with leukocyte detection capabilities. The process step 3316, the output of the module 3315, includes the locations in the video at which a leukocyte is detected, and the confidence score of the detection. Depending on the model, the leukocyte may also be classified according to its subtype (e.g., granulocytes, lymphocytes).

### M07. Establish Final Leukocyte Count Per Flow Volume and Report Findings

The modules 3315 and 3317 confirm the presence of candidate leukocytes identified in module 3312 by the “detect-and-link” method of particle tracking, and ensures the accuracy of the final leukocyte count. Module 3317 is concerned with “link”.

State estimation addresses the scenario in which leukocytes are intermittently concealed in the video by other cells. The number of particle tracks is reported as the final leukocyte count in step 3318, and dividing that number by the blood flow volume 3311 is the final leukocyte count per blood flow volume. The reporting finishes the process 3300 at step 3319.

### References

1. Germain, R.N., Robey, E.A. & Cahalan, M.D., A Decade of Imaging Cellular Motility and Interaction Dynamics in the Immune System. Science 336, 1676-1681 (2012).
2. Vestweber, D., How leukocytes cross the vascular endothelium. Nat. Rev. Immunol. 15, 692-704 (2015).
3. Springer, T.A., Traffic signals for lymphocyte recirculation and leukocyte emigration: The multistep paradigm. Cell 76, 301-314 (1994).
4. von Andrian, U.H. et al., Two-step model of leukocyte-endothelial cell interaction in inflammation: distinct roles for LECAM-1 and the leukocyte beta 2 integrins in vivo. Proc. Natl. Acad. Sci. 88, 7538-7542 (1991).
5. Sahu, A. et al., In vivo tumor immune microenvironment phenotypes correlate with inflammation and vasculature to predict immunotherapy response. Nat. Commun. 13, 5312 (2022).
6. Fabian-Jessing, B.K. et al., In vivo quantification of rolling and adhered leukocytes in human sepsis. Crit. Care 22, 240 (2018).
7. Uz, Z., Ince, C., Shen, L., Ergin, B. & van Gulik, T.M., Real-time observation of microcirculatory leukocytes in patients undergoing major liver resection. Sci. Rep. 11, 4563 (2021).
8. Massey, M.J. & Shapiro, N.I., A guide to human in vivo microcirculatory flow image analysis. Crit. Care 20, 35 (2016).
9. Ince, C. et al., Second consensus on the assessment of sublingual microcirculation in critically ill patients: results from a task force of the European Society of Intensive Care Medicine. Intensive Care Med. 44, 281-299 (2018).
10. Ulrich, M., Lange-Asschenfeldt, S. & Gonzalez, S., The use of reflectance confocal microscopy for monitoring response to therapy of skin malignancies. Dermatol. Pract. Concept. 2, 0202a10 (2012).
11. Rajadhyaksha, M., González, S., Zavislan, J.M., Rox Anderson, R. & Webb, R.H., In Vivo Confocal Scanning Laser Microscopy of Human Skin II: Advances in Instrumentation and Comparison With Histology11The authors have declared conflict of interest. J. Invest. Dermatol. 113, 293-303 (1999).
12. Wu, C.-H. et al., Imaging Cytometry of Human Leukocytes with Third Harmonic Generation Microscopy. Sci. Rep. 6, 37210 (2016).
13. Li, C. et al., Imaging leukocyte trafficking in vivo with two-photon-excited endogenous tryptophan fluorescence. Opt. Express 18, 988-999 (2010).
14. Ford, T.N., Chu, K.K. & Mertz, J., Phase-gradient microscopy in thick tissue with oblique back-illumination. Nat. Methods 9, 1195-1197 (2012).
15. Nomarski G., Differential microinterferometer with polarized waves. J Phys Radium Paris 16, 9S (1955).
16. Hamilton, D.K. & Sheppard, C.J.R., Differential phase contrast in scanning optical microscopy. J. Microsc. 133, 27-39 (1984).
17. Bagramyan, A. et al., Focus-tunable microscope for imaging small neuronal processes in freely moving animals. Photonics Res. 9, 1300-1309 (2021).
18. Huang, L., Mckay, G.N. & Durr, N.J., A Deep Learning Bidirectional Temporal Tracking Algorithm for Automated Blood Cell Counting from Non-invasive Capillaroscopy Videos. in Medical Image Computing and Computer Assisted Intervention—MICCAI 2021 (eds. de Bruijne, M. et al.) 415-424 (Springer International Publishing, 2021). doi:10.1007/978-3-030-87237-3_40.
   The citation of any document or reference is not to be construed as an admission that it is prior art with respect to the present invention.

Thus, the present invention provides a system and methods for imaging of microvasculature of tissue of a subject, and more particularly to a system and methods for stabilized noninvasive imaging of microvasculature in the oral mucosa.

In light of the principles and example embodiments described and illustrated herein, it will be recognized that the example embodiments can be modified in arrangement and detail without departing from such principles. Also, the foregoing discussion has focused on particular embodiments, but other configurations are also contemplated. In particular, even though expressions such as “in one embodiment”, “in another embodiment”, “in other embodiments”, “in some embodiments”, or the like are used herein, these phrases are meant to generally reference embodiment possibilities, and are not intended to limit the invention to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another.

Although the invention has been described in considerable detail with reference to certain embodiments, one skilled in the art will appreciate that the present invention can be used in alternative embodiments to those described, which have been presented for purposes of illustration and not of limitation. Therefore, the scope of the appended claims should not be limited to the description of the embodiments contained herein.

What is claimed is:

1. A system for imaging of microvasculature of tissue of a subject, the system comprising:
(a) a tissue stabilizer structured to contact the tissue of the subject to maintain a position of the region of the microvasculature being imaged; and
(b) an imaging instrument including:
(i) a housing having an imaging section,
(ii) an illumination device having a light-outputting end positioned in the imaging section of the housing for illuminating a region of the microvasculature with light, wherein the light-outputting end is offset relative to an optical axis of the imaging section,
(iii) an objective lens positioned in the imaging section of the housing such that the objective lens receives at least a portion of light scattered by the region of the microvasculature, and
(iv) an image detector positioned in the imaging section of the housing such that the image detector receives light redirected by the objective lens and detects microscopic images of the region of the microvasculature.

2. The system of claim 1 wherein:
the tissue stabilizer comprises a base, a sliding mechanism mounted on the base, an adapter for contacting the tissue, the adapter being mounted on the sliding mechanism, and
the adapter is moveable toward and away from the base.

3. The system of claim 2 wherein:
the base comprises a chin holder, and
the tissue stabilizer further comprises a frame, the chin holder and a forehead holder being mounted on the frame.

4. The system of claim 2 wherein:
the adapter includes a patterned surface finish for contacting the tissue.

5. The system of claim 2 wherein:
the adapter includes opposed tabs for contacting the tissue.

6. The system of claim 2 wherein:
the adapter applies mechanical pressure on edges of the oral mucosa tissue, at a distance of at least two millimeters from imaging regions of interest to minimize impact of the mechanical pressure on an imaging area.

7. The system of claim 2 wherein:
the adapter is bendable.

8. The system of claim 2 wherein:
the adapter is rigid.

9. The system of claim 2 wherein:
the adapter includes single or multiple light sources.

10. The system of claim 2 wherein:
the adapter includes single or multiple optical elements.

11. The system of claim 2 wherein:
the adapter includes a vacuum system.

12. The system of claim 5 wherein:
the adapter further includes a transparent sheet mounted between the opposed tabs.

13. The system of claim 1 wherein:
the tissue stabilizer comprises a base, a sliding mechanism mounted on the base, an adapter for contacting the tissue, the adapter being mounted on the sliding mechanism, and
the adapter is moveable laterally with respect to the base.

14. The system of claim 1 wherein:
the adapter is dimensioned for contacting oral mucosa of the subject.

15. The system of claim 1 wherein:
the adapter is dimensioned for contacting a lip of the subject.

16. The system of claim 2 wherein:
the adapter comprises a rod mounted between opposed connectors, the rod being dimensioned for contacting the tissue.

17. The system of claim 1 wherein:
the adapter comprises a flexible loop, the rod being dimensioned for contacting the tissue.

18. The system of claim 1 wherein:
the objective lens is a microlens.

19. The system of claim 1 wherein:
the objective lens is a gradient index (GRIN) objective lens.

20. The system of claim 1 wherein:
the objective lens is a gradient index (GRIN) objective lens, and
the system further comprises a doublet achromat lens.

21. The system of claim 1 wherein:
the image detector is a camera.

22. The system of claim 1 wherein:
the image detector is a CMOS sensor.

23. The system of claim 1 wherein:
the image detector is moveable with respect to the objective lens.

24. The system of claim 1 wherein:
the image detector detects microscopic images using oblique back-illumination (OBM).

25. The system of claim 1 wherein:
the image detector detects microscopic images using offset trans-illumination (OTM).

26. The system of claim 1 wherein:
the imaging section further comprises a vacuum device for stabilizing the tissue being imaged.

27. The system of claim 1 further comprising:
the imaging section further comprises an irrigation channel for supplying a fluid to keep the tissue being imaged moist.

28. The system of claim 1 wherein:
the illumination device comprises a light source and an optical fiber having the light-outputting end.

29. The system of claim 1 wherein:
the imaging section further comprises a sterile disposable unit.

30. The system of claim 29 wherein:
the sterile disposable unit includes single or multiple optical element(s), a light source, an irrigation channel, and a vacuum cavity.

31. The system of claim 1 wherein:
the imaging section further comprises an imaging tip that contains the objective lens, a vacuum device, an irrigation channel, and an illumination fiber of the illumination device, and
the objective lens is a microlens.

32. The system of claim 1 wherein:
the imaging section further comprises an imaging tip that contains the objective lens, a vacuum device, an irrigation channel, and an illumination fiber of the illumination device, and
the objective lens is gradient index (GRIN) lens.

33. The system of claim 32 wherein:
the imaging tip is disposable.

34. The system of claim 1 wherein:
the microscopic images include images of leukocyte-endothelial interaction in the microvasculature.

35. The system of claim 34 wherein:
the imaging is label-free imaging.

36. The system of claim 1 wherein:
the microscopic images are phase-gradient contrast images.

37. The system of claim 1 wherein:
the illumination device comprises a light source and an optical fiber having the light-outputting end, and
the light source comprises a light-emitting diode.

38. The system of claim 1 wherein:
the imaging is at a frame rate of 1 Hz to 1000 Hz.

39. The system of claim 1 wherein:
the imaging is at a frame rate of 1 Hz to 300 Hz.

40. The system of claim 1 wherein:
injected light power is automatically adjusted by a controller to prevent pixel(s) saturation of a data acquisition element.

41. The system of claim 1 wherein:
scattered light collection time of a data acquisition element is automatically adjusted by software to prevent pixel(s) saturation.

42. The system of claim 1 wherein:
the microscopic images include images of leukocytes in the microvasculature, and
the system further comprises a controller in electrical communication with the illumination device and the image detector, the controller being configured to execute a program stored in the controller to:
(i) receive the microscopic images from the image detector, and
(ii) use automated frame-by-frame leukocyte tracking to calculate average rolling velocity of the leukocytes in the microvasculature.

43. The system of claim 41 wherein the controller executes the program stored in the controller to:
(iii) compare the average rolling velocity of the leukocytes in the microvasculature to an average rolling velocity of leukocytes in heathy tissue.

44. A system for imaging of microvasculature of tissue of a subject, the system comprising:
an imaging instrument including a housing having an imaging section;
a tissue stabilizer structured to contact the tissue of the subject to maintain a position of the region of the microvasculature being imaged by the imaging instrument;
an illumination device having a light-outputting end positioned in the tissue stabilizer for illuminating a region of the microvasculature with light;
an objective lens positioned in the imaging section of the housing such that the objective lens receives at least a portion of light scattered by the region of the microvasculature; and
an image detector positioned in the imaging section of the housing such that the image detector receives light redirected by the objective lens and detects microscopic images of the region of the microvasculature.

45. The system of claim 44 wherein:
the tissue stabilizer comprises a base, a sliding mechanism mounted on the base, an adapter for contacting the tissue, the adapter being mounted on the sliding mechanism, and
the adapter is moveable toward and away from the base.

46. The system of claim 45 wherein:
the light-outputting end of the illumination device is positioned in the adapter.

47. The system of claim 45 wherein:
the base comprises a chin holder, and
the light-outputting end of the illumination device is positioned in the chin holder.

48. A system for imaging of microvasculature of tissue of a subject, the system comprising:
a tissue stabilizer structured to contact the tissue of the subject to maintain a position of the region of the microvasculature being imaged;
an illumination device having a light-outputting end positioned in the tissue stabilizer for illuminating a region of the microvasculature with light;
an objective lens positioned in the tissue stabilizer such that the objective lens receives at least a portion of light scattered by the region of the microvasculature; and
an image detector positioned in the tissue stabilizer such that the image detector receives light redirected by the objective lens and detects microscopic images of the region of the microvasculature.

49. The system of claim 48 wherein:
the tissue stabilizer comprises a first arm and an opposed second arm, the first arm and the second arm defining a space therebetween for receiving the tissue, and
the illumination device, the objective lens, and the image detector are arranged on the first arm such that the image detector detects microscopic images using oblique back-illumination (OBM).

50. The system of claim 48 wherein:
the tissue stabilizer comprises a first arm and an opposed second arm, the first arm and the second arm defining a space therebetween for receiving the tissue, and
the objective lens and the image detector are arranged on the first arm, and the illumination device is arranged on the second arm such that the image detector detects microscopic images using offset trans-illumination (OTM).

51. The system of claim 48 wherein:
the tissue stabilizer comprises a first arm, an opposed second arm, and a hinge connecting the first arm and the second arm such that a variable size space is created between the first arm and the second arm for receiving the tissue.

52. A system for imaging of microvasculature of tissue of a subject, the system comprising:
an imaging instrument operable to capture an image;
an electronic processor in communication with the imaging instrument, the electronic processor being configured to execute a program stored in the electronic processor to:
receive the image from the imaging instrument; and
reduce a foreground of the image to a skeleton that captures one or more attributes of the foreground including at least one of curvature, connectivity, and extent wherein the skeleton defines a transformed coordinate system for quantifying one or more perfusion parameters in the microvasculature.

53. The system of claim 52 wherein:
the skeleton is a line that follows an axis of a vessel of the microvasculature and bends in accordance with local curvature of the vessel.

54. The system of claim 52 wherein the electronic processor executes the program stored in the electronic processor to:
create a transformed coordinate system by generating multiple gridlines to cover a full width of a region of interest (ROI) of microvasculature.

55. The system of claim 54, wherein the electronic processor executes the program stored in the electronic processor to:
create the transformed coordinate system such that two axes run parallel and normal to blood flow, respectively, wherein an axis parallel to the blood flow is defined by the skeleton, and an axis normal to the blood flow is defined by normal lines of the skeleton.

56. The system of claim 54, wherein the electronic processor executes the program stored in the electronic processor to:
create a collection of skeleton and lines created in reference to the skeleton defining an x′-axis and y′-gridlines of the transformed coordinate system, the y′-gridlines running in the direction of blood flow of microvascular ROI.

57. The system of claim 56, wherein the y′-gridlines of the transformed coordinate system have a same pixel length, regardless of curvature of the ROI.

58. The system of claim 54, wherein the electronic processor executes the program stored in the electronic processor to:
draw a space-time diagram for each y′-gridline at each time segment and vessel block, the vessel block being defined as a unit for length along an axis of microvascular ROI.

59. The system of claim 58, wherein the electronic processor executes the program stored in the electronic processor to:
calculate a blood flow velocity by consolidating multiple space-time diagrams of individual y′-gridline, time segment and vessel block.

60. The system of claim 59, wherein the electronic processor executes the program stored in the electronic processor to:
calculate blood flow volume rate by multiplying the blood flow velocity and a cross-section area of the ROI.

61. The system of claim 58, wherein the electronic processor executes the program stored in the electronic processor to:
calculate a count of leukocytes by summing along slopes of the space-time diagrams to generate an intensity profile wherein the intensity profiles are further consolidated from multiple y′-gridlines, time segments and vessel blocks such that a number of peaks in a consolidated intensity profile gives an estimate of the count of leukocytes.

62. The system of claim 59, wherein the consolidation is performed by dynamic time warping to peak match an intensity profile of vessel blocks, while allowing variations in time delay among candidate leukocytes.

63. The system of claim 52, wherein the electronic processor executes the program stored in the electronic processor to:
estimate a time of appearance of candidate leukocytes by determining a peak position in an intensity profile.

64. The system of claim 52, wherein the electronic processor executes the program stored in the electronic processor to:
gate an approximate space and time of appearance of candidate leukocytes in the video using a consolidated intensity profile.

65. A system for imaging of microvasculature of tissue of a subject, the system comprising:
an imaging instrument operable to capture an image;
an electronic processor in communication with the imaging instrument, the electronic processor being configured to execute a program stored in the electronic processor to:
receive the image from the imaging instrument;
access a deep learning model that has been trained on training data to detect perfusion and leukocyte feature data from the image input; and
apply the image to the machine learning model to quantify one or more perfusion parameters in the microvasculature.

66. The system of claim 65, wherein the deep learning model is a neural network.

67. The system of claim 66, wherein the neural network is a convolutional neural network.

68. The system of claim 65, wherein the machine learning model is applied to gated spatial regions and time that contain candidate leukocytes.

69. The system of claim 65, wherein the electronic processor executes the program stored in the electronic processor to:
detect coordinates of the image at which a leukocyte is detected, and a probability score of the detection.

70. A method for in vivo flow cytometry of a biological fluid in a subject, the method comprising:
(a) contacting tissue of the subject with a tissue stabilizer to maintain a position of a biological structure of the subject;
(b) providing, using an illumination device, light to a portion of a region of the biological structure to continuously illuminate the region of the biological structure;
(c) continuously detecting, using an image detector, microscopic images from the region of the biological structure based on light scattered by the biological structure of the subject, wherein illumination is at an oblique angle due to offset geometry of the illumination device; and
(d) analyzing the microscopic images to identify characteristics of a biological fluid in the biological structure.

71. The method of claim 70 wherein:
step (c) comprises detecting the microscopic images comprises producing optical images through oblique back-illumination microscopy (OBM).

72. The method of claim 70 wherein:
step (c) comprises detecting the microscopic images comprises producing optical images through offset trans-illumination (OTM).

73. The method of claim 70 wherein:
the biological structure is microvasculature of the subject; and
step (d) comprises quantifying one or more perfusion parameters in the microvasculature.

74. The method of claim 70 wherein:
the biological structure is microvasculature of the subject; and
step (d) comprises quantifying a count of leukocytes in the microvasculature.

75. The method of claim 70 wherein:
step (c) comprises detecting the microscopic images without a label.

76. The method of claim 70 wherein:
step (c) comprises detecting the microscopic images at a frame rate of 1 Hz to 1000 Hz.

77. The method of claim 70 wherein:
the biological structure is microvasculature of the subject; and
step (d) comprises using automated frame-by-frame leukocyte tracking to calculate average rolling velocity of leukocytes in the microvasculature.

78. The method of claim 70 wherein:
the biological structure is microvasculature of the subject; and
step (d) comprises reducing a foreground of each microscopic image to a skeleton that captures one or more attributes of the foreground including at least one of curvature, connectivity, and extent, wherein the skeleton defines a transformed coordinate system for quantifying one or more perfusion parameters in the microvasculature.

79. The method of claim 70 wherein:
the biological structure is microvasculature of the subject; and
step (d) further comprises creating a transformed coordinate system by generating multiple gridlines to cover a full width of a region of interest (ROI) of microvasculature.

80. The method of claim 79 wherein:
the biological structure is microvasculature of the subject; and
step (d) further comprises creating the transformed coordinate system in which two axes run parallel and normal to blood flow, respectively, wherein an axis parallel to the blood flow is defined by the skeleton, and an axis normal to the blood flow is defined by normal lines of the skeleton.

81. The method of claim 70 wherein:
the biological structure is microvasculature of the subject; and
step (d) comprises creating a transformed coordinate system wherein two axes run parallel and normal to the blood flow, respectively.

82. The method of claim 81 wherein:
step (d) comprises creating the transformed coordinate system wherein an axis parallel to the blood flow is defined by the skeleton, and an axis normal to the blood flow is defined by normal lines of the skeleton.

83. The method of claim 79 wherein:
the biological structure is microvasculature of the subject; and
step (d) comprises drawing a space-time diagram for each y′-gridline at each time segment and vessel block, the vessel block being defined as a unit for length along an axis of microvascular ROI.

84. The method of claim 83 wherein:
step (d) further comprises calculating a blood flow velocity by consolidating multiple space-time diagrams of individual y′-gridline, time segment and vessel block.

85. The method of claim 77 wherein:
the biological structure is microvasculature of the subject; and
step (d) comprises accessing a deep learning model that has been trained on training data to detect perfusion and leukocyte feature data from the image input; and applying the image to the machine learning model to quantify one or more perfusion parameters in the microvasculature.
